(19) United States
(12) Patent Application Publication
(10) Pub. No.: US 2026/0284404 A1
(43) Pub. Date: Sep. 24, 2026

(54) CONTROLLING ELECTRICAL STIMULATION BY A MEDICAL DEVICE

(71) Applicant: Medtronic, Inc., Minneapolis, MN (US)

(72) Inventors: Todd D. Zenisek, Georgetown, TX (US); Andrew J. Cleland, Macquarie Park, New South Wales (AU)

(21) Appl. No.: 19/479,371

(22) PCT Filed: Apr. 11, 2024
(86) PCT No.: PCT/IB2024/053543

§ 371 Date: Oct. 28, 2025

Related U.S. Application Data
(60) Provisional application No. 63/499,059, filed on Apr. 28, 2023.

(51) Int. Cl. A61N 1/36

(52) U.S. Cl. CPC A61N 1/36139

(57) ABSTRACT

A system including processing circuitry configured to: receive, via sensing circuitry of the system, a first signal representative of a first evoked electrical signal elicited from electrical stimulation delivered during a posture state of the patient; determine a first signal characteristic of the first evoked electrical signal; receive, via one or more user devices of the system, patient input indicative of a comfort level of the patient associated with the first evoked electrical signal; determine, based on the first signal characteristic and the patient input, a target characteristic for the posture state; receive, via the sensing circuitry, a second signal representative of a second evoked electrical signal elicited from the electrical stimulation delivered during the posture state; and control, based on a second signal characteristic of the second evoked electrical signal and the target characteristic, one or more parameters that at least partially define subsequent delivery of the electrical stimulation.

This Application claims priority from U.S. Provisional Patent Application 63/499,059, filed 28 Apr. 2023, the entire content of which is incorporated herein by reference.

### TECHNICAL FIELD

This disclosure generally relates to electrical stimulation therapy, and more specifically, control of electrical stimulation therapy.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

### SUMMARY

In general, the disclosure is directed to devices, systems, and techniques for controlling electrical stimulation therapy based at least in part on evoked signals (e.g., evoked compound action potentials (ECAPs) elicited by an electrical stimulation signal in a posture state of a patient.

A medical device (e.g., an implantable medical device) can deliver one or more electrical stimulation signals to the patient via one or more leads, and the medical device may sense ECAPs elicited by the electrical stimulation signals. The medical device may receive patient input indicating a posture of the patient and/or a level of pain perceived by the patient in that patient. In some examples, the medical device may determine the posture of the patient based on sensor data of one or more sensors coupled to the processing circuitry of the medical device and associate patient input indicative of the level of pain or other patient perceived feedback to that detected posture state.

The medical device may be configured to compare, when the patient is in a same posture state, currently sensed evoked signals against prior evoked signals and corresponding patient input for that same posture state. Based on the comparison between the current and prior evoked signals, the medical device may adjust stimulation parameters of the electrical stimulation signal to optimize treatment of perceived pain or discomfort that may be experienced by the patient in that posture state and in other posture states. In some examples, the medical device may compare the evoked signals to perform different functions including, but are not limited to, detecting damage and/or migration of electrical leads coupled to the medical device and configured to deliver the electrical stimulation signals, determining changes in a disease state of the patient, determining effects of one or more medications or therapeutic substances on the patient, or any combination thereof.

In some examples, the disclosure describes a system comprising: processing circuitry configured to: receive, via sensing circuitry of the system, a first signal representative of one or more first evoked electrical signals sensed from tissue of a patient and elicited from electrical stimulation delivered during a posture state of the patient; determine a first signal characteristic of the one or more first evoked electrical signals for the posture state; receive, via one or more user devices of the system, patient input indicative of a comfort level of the patient associated with the one or more first evoked electrical signals during the posture state; determine, based on the first signal characteristic and the patient input, a target characteristic for the posture state; receive, via the sensing circuitry, a second signal representative of one or more second evoked electrical signals sensed from the tissue of the patient and elicited from the electrical stimulation delivered during the posture state; and control, based on a second signal characteristic of the one or more second evoked electrical signals and the target characteristic, one or more parameters that at least partially define subsequent delivery of the electrical stimulation to the patient during the posture state.

In some examples, the disclosure describes a method comprising: receiving, by processing circuitry of a medical device system and via sensing circuitry of the medical device system, a first signal representative of one or more first evoked electrical signals sensed from tissue of a patient and elicited from electrical stimulation delivered during a posture state of the patient; determining, by the processing circuitry, a first signal characteristic of the one or more first evoked electrical signals for the posture state; receiving, by the processing circuitry from one or more user devices, patient input indicative of a comfort level of the patient associated with the one or more first evoked electrical signals during the posture state; determining, by the processing circuitry and based on the first signal characteristic and the patient input, a target characteristic for the posture; receiving, by the processing circuitry and via the sensing circuitry, a second signal representative of one or more second evoked electrical signals sensed from the tissue of the patient and elicited from the electrical stimulation delivered during the posture state; and controlling, by the processing circuitry and based on a second signal characteristic of the one or more second evoked electrical signals and the target characteristic, one or more parameters that at least partially define subsequent delivery of the electrical stimulation to the patient during the posture state.

In some examples, the disclosure describes a computer-readable medium comprising instructions that, when executed, cause processing circuitry of an implantable medical device system to perform a method comprising: receiving, by processing circuitry of a medical device system and via sensing circuitry of the medical device system, a first signal representative of one or more first evoked electrical signals sensed from tissue of a patient and elicited from electrical stimulation delivered during a posture state of the patient; determining, by the processing circuitry, a first signal characteristic of the one or more first evoked electrical signals for the posture state; receiving, by the processing circuitry from one or more user devices, patient input indicative of a comfort level of the patient associated with the one or more first evoked electrical signals during the posture state; determining, by the processing circuitry and based on the first signal characteristic and the patient input, a target characteristic for the posture; receiving, by the processing circuitry and via the sensing circuitry, a second signal representative of one or more second evoked electrical signals sensed from the tissue of the patient and elicited from the electrical stimulation delivered during the posture state; and controlling, by the processing circuitry and based on a second signal characteristic of the one or more second evoked electrical signals and the target characteristic, one or more parameters that at least partially define subsequent delivery of the electrical stimulation to the patient during the posture state.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) configured to deliver stimulation therapy to a patient and an external programmer, in accordance with one or more techniques of this disclosure.

FIG. 2 is a block diagram illustrating an example configuration of components of an IMD of FIG. 1, in accordance with one or more techniques of this disclosure.

FIG. 3 is a block diagram illustrating an example configuration of components of the external programmer of FIG. 1, in accordance with one or more techniques of this disclosure.

FIG. 4 is a graph illustrating example evoked compound action potentials (ECAPs) sensed for respective stimulation pulses, in accordance with one or more techniques of this disclosure.

FIG. 5 is an example timing diagram illustrating an example of electrical stimulation pulses, respective stimulation pulses, and respective sensed ECAPs, in accordance with one or more techniques of this disclosure.

FIG. 6A is a conceptual diagram illustrating a spine of a patient with an implanted electrical lead in a first posture state, in accordance with the one or more techniques of this disclosure.

FIG. 6B is a conceptual diagram illustrating the spine of FIG. 6A in a second posture state, in accordance with the one or more techniques of this disclosure.

FIG. 7 is a flow diagram illustrating an example process for delivering stimulation therapy to a patient based on a sensed evoked signal and a patient posture state, in accordance with one or more techniques of this disclosure.

FIG. 8 is a flow diagram illustrating an example process for controlling stimulation therapy based on sensed evoked signals and a patient posture state, in accordance with one or more techniques of this disclosure.

FIG. 9 is a flow diagram illustrating an example process for determining damage and/or migration of an electrical lead within the body of the patient based on a sensed evoked signal and a patient posture state, in accordance with one or more techniques of this disclosure.

Like reference characters denote like elements throughout the description and figures.

### DETAILED DESCRIPTION

The disclosure describes example medical devices, systems, and techniques for controlling electrical stimulation therapy delivered to a patient based at least in part on evoked electrical signals (e.g., evoked compound action potentials (ECAPs)) sensed or detected by a medical device in a determined posture state of a patient. Electrical stimulation therapy is typically delivered by a medical device to a target tissue (e.g., one or more nerves or muscle) of a patient via two or more electrodes. Parameters of the electrical stimulation therapy (e.g., electrode combination, voltage or current amplitude, pulse width, pulse frequency, etc.) are selected by a clinician, the patient, and/or the medical device to provide relief from various symptoms, such as pain, muscle disorders, etc.

ECAPs are a measure of neural recruitment because each ECAP signal represents the superposition of electrical potentials generated from axons firing in response to an electrical stimulus (e.g., a stimulation pulse). Changes in a characteristic (e.g., an amplitude of a portion of the signal, an area under one or more peaks, frequency content, and/or maximum and/or minimum peak timing) of an ECAP signal occur as a function of how many axons have been activated by the delivered stimulation pulse. A system can monitor changes in the characteristic of the ECAP signal and use that change in the characteristic to adjust one or more stimulation parameters that at least partially defines the stimulation pulses delivered to the patient. For example, the system can reduce the intensity of stimulation pulses (e.g., reduce a current amplitude and/or pulse width) in response to detecting an increase in an amplitude of an ECAP signal. ECAPs may vary based on a posture state of a patient. In some examples, changes in the ECAP signal may be due to changes in the posture state of the patient and may and the changes in the ECAP signals alone may be insufficient to determine a need to adjust parameters of the electrical stimulation therapy. Moreover, it may be difficult to track progression of disease or changes to the patient therapy over time by only monitoring ECAP signals.

In some examples, the medical device may deliver stimulation pulses in the form of control pulses and informed pulses. More specifically, electrical stimulation pulses are delivered in the form of informed pulses and control pulses that are at least partially interleaved with each other. Control pulses (e.g., stimulation signal test pulses) are those stimulation pulses that are configured to elicit one or both of a stimulation signal and a detectable ECAP signal. In some examples, control pulses may contribute to the therapy for a patient. In other examples, control pulses do not contribute to the therapy for the patient, e.g., non-therapeutic pulses. In this manner, control pulses may or may not be configured to elicit a therapeutic effect for the patient. Informed pulses are those stimulation pulses that are at least partially defined by one or more parameters based on the detectable stimulation signal elicited from one or more control pulses. In some examples, one or more informed pulses are at least partially defined by one or more parameters based on a respective ECAP elicited from one or more control pulses. In this manner, the informed pulses are “informed” by the ECAP signal detected from a control pulse. Informed pulses are also configured to provide a therapy to a patient, such as paresthesia that relieves pain symptoms.

As described herein, a medical device may sense evoked signals (e.g., ECAP signals) elicited by electrical stimulation delivered to the tissue of the patient when the patient is in a particular posture state (e.g., standing, sitting, crouching, lying down). The medical device may determine the posture state based on sensed signals and/or receive patient input identifying the current posture state. The medical device may further receive patient input (e.g., via an external programmer or via interaction with the medical device through tapping or other modality) indicating patient comfort, or discomfort, associated (e.g., correlated) with the sensed evoked signals. Based on the sensed evoked signals and the patient input, the medical device and/or a medical device system may correspond different levels of evoked signal characteristic(s) to different comfort levels perceived by the patient. In some examples, a particular level of an evoked signal characteristic correlated with a lack of discomfort perceived by the patient may be established as a target value or target range for the evoked signal characteristic in a baseline evoked electrical signal of the patient in the particular posture state. Each posture state, or each set of one or more posture states, may be correlated with the target value or target range for the evoked signal characteristic.

The medical device may subsequently sense other evoked signals in response to the electrical stimulation therapy. The medical device and/or medical device system may compare evoked signals and adjust parameters of the electrical stimulation therapy based on deviations of the signal characteristic(s) of the subsequent sensed evoked signals from the signal characteristics of a baseline evoked signal. The medical device may also monitor for patient input of comfort levels and corresponding evoked signal characteristic values over time to adjust the target value or target range of the evoked signal characteristic value.

The techniques of this disclosure may provide one or more advantages. For example, adjusting parameters of the stimulation therapy based on the sensed evoked signals for a given patient posture state may reduce an amount of patient input required to adjust the stimulation therapy and/or reduce an amount of time required to adjust the stimulation therapy, thereby simplifying the process required to alleviate, via the stimulation therapy, discomfort perceived by the patient. In some examples, adjusting the parameters based on the sensed evoked signals and the posture state may increase optimization of the delivery of the stimulation therapy (e.g., to a minimum effective dose), thereby reducing power consumption and increasing an operational lifespan of a medical device. In some examples, the use of the sensed evoked signals and the posture state to control stimulation therapy may allow a medical device and/or medical device system to avoid unnecessary adjustments of the stimulation therapy due to acute discomfort perceived by the patient, thereby reducing patient discomfort, e.g., due to unnecessary changes in the stimulation therapy. In some examples, a medical device and/or medical device system may use the sensed evoked signals and the patient posture state to detect possible damage, migration of electrical leads coupled to a medical device, and/or determine changes to the disease state of the patient, thereby improving responsiveness to the likelihood of damage to a medical device.

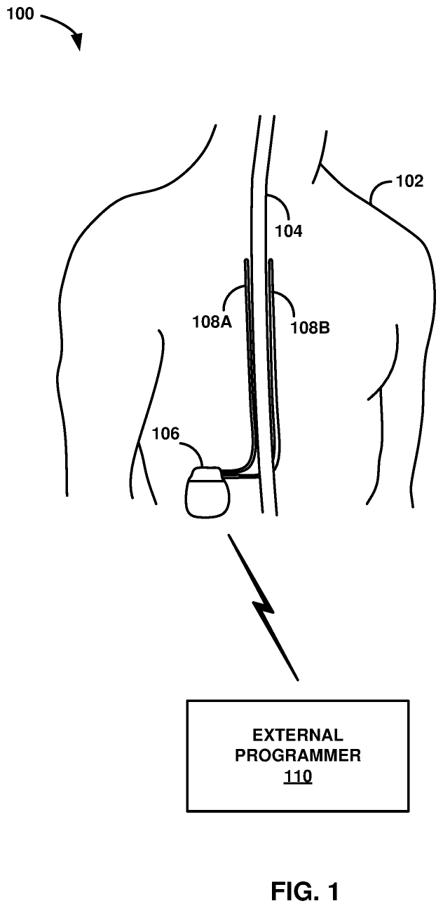

FIG. 1 is a conceptual diagram illustrating an example system 100 that includes an implantable medical device (IMD) 106 While this disclosure is described primarily with reference to one IMD 106, other examples may include two or more IMDs 106. IMD 106 may deliver electrical stimulation pulses to tissue of patient 102, e.g., via leads 108A, 108B (collectively referred to herein as “leads 108”). IMD 106 may be configured to sense evoked signals (e.g., ECAPs) or other bioelectrical signals, sense a posture of patient 102, deliver other medical therapy to patient 102 (e.g., a therapeutic substance via a pump), and/or perform any other medical functions.

As illustrated in FIG. 1, IMD 106 is configured to deliver spinal cord stimulation (SCS) therapy to nerves on or adjacent to a spinal cord 104 of patient 102. In some examples, a plurality of IMDs 106 may be configured to deliver SCS therapy and/or PNS therapy to patient 102. For example, one IMD may deliver “priming” stimulation pulses to glial cells of spinal cord 104 and another IMD 106 may deliver “base” stimulation pulses to a peripheral nerve as a part of a SCS therapy to reduce pain experienced by patient 102. In such examples, the base stimulation pulses may generate action potentials (e.g., ECAPs) in the peripheral nerves which may be detected by IMD 106.

IMD 106 may be configured to communicate with an external programmer 120, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices.

As shown in FIG. 1, system 100 IMD 106, leads 108, and external programmer 120 shown in conjunction with a patient 102, who is ordinarily a human patient. In the example of FIG. 1, IMD 106 is an implantable electrical stimulator that is configured to generate and deliver electrical stimulation therapy to patient 102 via one or more electrodes of electrodes of leads 108A and/or 108B, e.g., for relief of chronic pain or other symptoms. In some examples, IMD 106 may be coupled to a single lead 108 carrying multiple electrodes or two or more leads 108, each carrying multiple electrodes. This electrical stimulation may be delivered in the form of a continuous stimulation signal or stimulation pulses. In some examples, IMD 106 may be configured to generate and deliver control pulses configured to elicit ECAP signals, e.g., to sense ECAP signals for a given posture state. The control pulses may or may not contribute to therapy in some examples. In some examples, IMD 106 may, in addition to control pulses, deliver informed pulses that contribute to the therapy for the patient, but which do not elicit detectable ECAP signals. IMD 106 may be a chronic electrical stimulator that remains implanted within patient 102 for weeks, months, or even years. In other examples, IMD 106 may be a temporary, or trial, stimulator used to screen or evaluate the efficacy of electrical stimulation for chronic therapy. IMD 106 may be implanted within patient 102 or coupled to percutaneously implanted leads (e.g., leads 108). In some examples, IMD 106 is leadless.

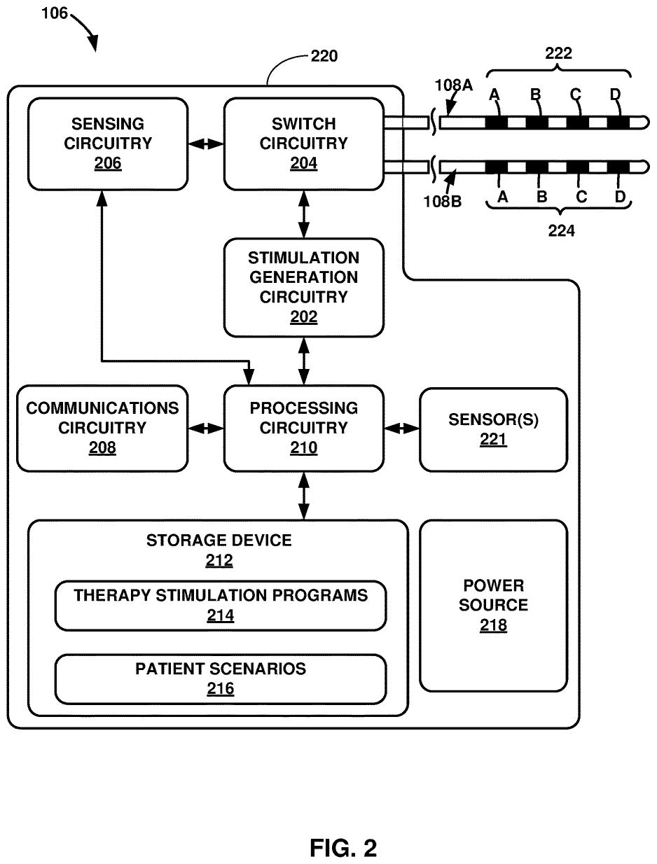

IMD 106 may be constructed of any polymer, metal, or composite material sufficient to house the components of IMD 106 (e.g., components illustrated in FIG. 2) within patient 102. In this example, IMD 106 may be constructed with a biocompatible housing, such as titanium or stainless steel, or a polymeric material such as silicone, polyurethane, or a liquid crystal polymer. IMD 106 may be surgically implanted at a site in patient 102 near the pelvis, abdomen, thigh, or buttocks. In other examples, IMD 106 may be implanted within other suitable sites within patient 102, which may depend, for example, on the target site within patient 102 for the delivery of electrical stimulation therapy (e.g., along spinal cord 104 of patient 102). The outer housing of IMD 106 may be configured to provide a hermetic seal for components, such as a rechargeable or non-rechargeable power source. In addition, in some examples, the outer housing of IMD 106 is selected from a material that facilitates receiving energy to charge the rechargeable power source.

Electrical stimulation energy, which may be constant current or constant voltage-based pulses, for example, is delivered from IMD 106 to one or more target tissue sites of patient 102 via one or more electrodes (not shown) of corresponding implantable leads 108. In the example of FIG. 1, each of leads 108 carry electrodes that are placed adjacent to the target tissue (e.g., near spinal cord 104). One or more of the electrodes may be disposed at a distal tip of each of leads 108 and/or at other positions at intermediate points along the lead. Leads 108 may be implanted and coupled to IMD 106. The electrodes may transfer electrical stimulation generated by an electrical stimulation generator in IMD 106 to tissue of patient 102. Although leads 108 may each be a single lead, leads 108 may include a lead extension or other segments that may aid in implantation or positioning of leads 108. In some examples, IMD 106 may be a leadless stimulator with one or more arrays of electrodes arranged on a housing of the stimulator rather than leads that extend from the housing. In addition, in some other examples, system 100 may include one lead or more than two leads, each coupled to IMD 106 and directed to similar or different target tissue sites.

The electrodes of leads 108 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 108 will be described for purposes of illustration.

The deployment of electrodes via leads 108 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 108 are linear leads having 8 ring electrodes along the axial length of the lead. In another example, the electrodes are segmented rings arranged in a linear fashion along the axial length of the lead and at the periphery of the lead.

The stimulation parameter of a therapy stimulation program that defines the electrical stimulation therapy delivered by IMD 106 through the electrodes of leads 108 may include information identifying which electrodes have been selected for delivery of stimulation according to a stimulation program, the polarities of the selected electrodes, i.e., the electrode combination for the program, and voltage or current amplitude, pulse frequency, pulse width, pulse shape of stimulation delivered by the electrodes. These stimulation parameters of stimulation pulses (e.g., control pulses and/or informed pulses) are typically predetermined parameter value(s) determined prior to delivery of the stimulation pulses (e.g., set according to a stimulation program). However, in some examples, system 100 changes parameter value(s) automatically based on one or more factors or based on patient input.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 102.

In some examples, leads 108 and/or IMD 106 includes one or more sensors configured to allow IMD 106 to monitor one or more parameters of patient 102, such as a posture state of patient 102. Posture states may include, but are not limited to, standing, sitting, slouching, crouching, lying down, or the like. When patient 102 is in each posture state, the spine and spinal cord 104 of patient 102 may be in a different position and IMD 106 may deliver electrical stimulation to different nerves and/or nerve fiber bundles of patient 102. The different delivery locations may cause IMD 106 to sense, in response to a same electrical stimulation therapy, ECAP signals having different signal characteristic for the different posture states. In some examples, IMD 106 may stimulate a same nerve and/or a same fiber bundle in different posture states. In such examples, IMD 106 may sense ECAP signals having same or similar signal characteristic(s) for the different posture states. In some examples, the one or more sensors of IMD 106 may sense, e.g., from nerves of patient 102, ECAP signals.

Each of IMDs 106 may be configured to deliver electrical stimulation therapy to patient 105 via selected combinations of electrodes carried by one or both of leads 108, alone or in combination with an electrode carried by or defined by an outer housing of IMD 106. The target tissue for the electrical stimulation therapy may be any tissue affected by electrical stimulation, which may be in the form of electrical stimulation pulses or continuous waveforms. In some examples, the target tissue includes nerves, smooth muscle or skeletal muscle. In the example illustrated by FIG. 1, e.g., with respect to IMD 106, the target tissue is tissue proximate spinal cord 104, such as within an intrathecal space or epidural space of spinal cord 104, or, in some examples, adjacent nerves that branch off spinal cord 104. Leads 108 (e.g., leads 108A, 108B) may be introduced into spinal cord 104 in via any suitable region, such as the thoracic, cervical or lumbar regions. Stimulation of spinal cord 104 may, for example, prevent pain signals from traveling through spinal cord 104 and to the brain of patient 102. Patient 102 may perceive the interruption of pain signals as a reduction in pain and, therefore, efficacious therapy results. In other examples, stimulation of spinal cord 104 may produce paresthesia which may be reduce the perception of pain by patient 102, and thus, provide efficacious therapy results.

A user, such as a clinician or patient 102, may interact with a user interface of an external programmer 120 to program and/or to control IMD 106. Programming of IMD 106 may refer generally to the generation and transfer of commands, programs, or other information to control the operation of IMD 106. In this manner, IMD 106 may receive the transferred commands and programs from external programmer 120 to control electrical stimulation therapy (e.g., informed pulses) and control stimulation (e.g., control pulses). For example, external programmer 120 may transmit therapy stimulation programs, stimulation parameter adjustments, therapy stimulation program selections, patient input, or other information to control the operation of IMD 106, e.g., by wireless telemetry or wired connection. As described herein, stimulation delivered to the patient may include control pulses, and, in some examples, stimulation may include control pulses and informed pulses.

In some cases, external programmer 120 may be characterized as a physician or clinician programmer if it is primarily intended for use by a physician or clinician. In other cases, external programmer 120 may be characterized as a patient programmer if it is primarily intended for use by a patient. A patient programmer may be generally accessible to patient 102 and, in many cases, may be a portable device that may accompany patient 102 throughout the patient's daily routine. For example, a patient programmer may receive input from patient 102 when the patient wishes to terminate or change electrical stimulation therapy, and/or may be configured to receive patient input indicating a comfort or discomfort level associated with therapy in that patient state. In general, a physician or clinician programmer may support selection and generation of programs by a clinician for use by IMD 106, whereas a patient programmer may support adjustment and selection of such programs by a patient during ordinary use. In other examples, external programmer 120 may include, or be part of, an external charging device that recharges a power source of IMD 106. In this manner, a user may program and charge IMD 106 using one device, or multiple devices.

As described herein, information may be transmitted between external programmer 120 and IMD 106. Therefore, IMD 106 and external programmer 120 may communicate via wireless communication using any techniques known in the art. Examples of communication techniques may include, for example, radiofrequency (RF) telemetry and inductive coupling, but other techniques are also contemplated. In some examples, external programmer 120 includes a communication head that may be placed proximate to the patient's body near the IMD 106 implant site to improve the quality or security of communication between IMD 106 and external programmer 120. Communication between external programmer 120 and IMD 106 may occur during power transmission or separate from power transmission. In some examples, IMD 106 and external programmer 120 may communicate via a intermediate device (e.g., another IMD, a body band, a wearable intermediary device such as a smartwatch or a smartphone).

In some examples, IMD 106, in response to commands from external programmer 120, delivers electrical stimulation therapy to a target tissue site in patient 102 via electrodes (not depicted) on leads 108. In some examples, IMD 106 modifies therapy stimulation programs as therapy needs of patient 102 evolve over time and/or based on changes in the sensed ECAP signals when patient 102 is in a same posture state. For example, the modification of the therapy stimulation programs may cause the adjustment of at least one parameter of the plurality of informed pulses. When patient 102 receives the same therapy for an extended period, the efficacy of the therapy may be reduced. In some cases, parameters of the plurality of informed pulses may be automatically updated.

In some examples, progression of a disease state, effects of a therapeutic substance, damage and/or migration of leads 108, onset of acute pain, changes to chronic pain, damage or deterioration of one or more nerves, and/or changes in discomfort perceived by patient 102 may cause changes in the sensed ECAP signals over time when patient 102 is in a same posture state. In such examples, based on the differences in signal characteristic(s) between a currently sensed evoked signal and prior evoked signals, system 100 (e.g., IMD 106, external programmer 120) may adjust parameters of the electrical stimulation therapy, e.g., until IMD 106 determines that signal characteristic(s) of the currently sensed evoked signal is within a target range(s) of a target characteristic based on the prior evoked signals. The signal characteristic of the ECAP signals may include, but is not limited to, a nerve conduction velocity of the signal, an amplitude of a portion of the signal (e.g., amplitude between two peaks), an area under one or more peaks, frequency content, and/or maximum and/or minimum peak timing.

In some examples, patient 102 may switch from a first posture state to a second posture state, e.g., from sitting to standing. In such examples, the electrical stimulation therapy configured for the first posture state may elicit ECAP signals having a signal characteristic outside of the target range(s) of the target characteristic for the second posture state. In such examples, system 100 may adjust the parameters of the electrical stimulation therapy until IMD 106 detects that the currently sensed ECAP signals have a signal characteristic within the target range(s)of the target characteristic for the second posture state. The target range or target characteristic value for the signal characteristic may be derived from previous ECAP signals received during a posture state in which patient input was provided indicating a comfort or discomfort level in that posture state.

In the example of FIG. 1, IMD 106 is described as performing a plurality of processing and computing functions. For example, IMD 106 may determine a current posture state of patient 102, receive the sensed ECAP signals corresponding to the electrical stimulation therapy, determine whether signal characteristic(s) of the sensed ECAP signal are within target range(s) of target characteristic(s) for the posture state, and adjust parameters of the electrical stimulation therapy to elicit ECAP signals having signal characteristic(s) within the target range(s). IMD 106 may perform the plurality of processing and computing functions in a closed-loop process, i.e., without additional input from external programmer 120, a clinician, or patient 102. In some examples, IMD 106 may receive the posture state of patient 102 and the target range(s) of target characteristic(s) for the posture state from an external computing device and/or system (e.g., from external programmer 120).

In some examples, as a disease state of a patient 102 progresses over time, the ECAP signal characteristic(s) for each posture state may change accordingly. IMD 106 may be configured to adjust, based on the changes in the disease state, the target range(s) of the target characteristic(s). Similarly, in some examples, use of particular therapeutic substances may affect physiological parameters of patient 102 (e.g., nerve conduction velocity) and, by extension, the ECAP signal characteristics. IMD 106 may be configured to detect, based on the changes in the ECAP signal characteristics, the presence of the effects of any therapeutic substances. Based on such determinations, IMD 106 may adjust the parameters of the electrical stimulation therapy to account for the effects of the therapeutic substance and/or adjust the target range(s) of the target characteristic(s) to account for the effects of the therapeutic substance. In addition, or alternatively, IMD 106 may detect changes to the disease status of the patient by receiving additional patient input changing the comfort or discomfort level for the therapy even if the signal characteristic remains within the target range.

In some examples, patient 102 may experience acute episodes of pain or discomfort. In other medical devices, patient input indicating the perception of an acute episode may cause the medical device to unnecessarily adjust parameters of the electrical stimulation therapy. Once the acute episode subsides, the medical device must then revert the adjustments to the parameters of the therapy. Such changes may not address the acute episodes of pain and may conversely increase discomfort of patient 102, e.g., due to the changes in the electrical stimulation therapy.

IMD 106 may, in response to the patient input indicating the perception of an acute episode of pain or discomfort, determine whether the sensed ECAP signals correspond to the patient input. If the sensed ECAP signals do not correspond to pain or discomfort, IMD 106 may maintain the current parameters of the electrical stimulation therapy. Maintaining the current parameters may reduce unnecessary power expenditure and/or reduce discomfort perceived by patient 102 after subsidence of the acute episode. In some examples, IMD 106 may temporarily deliver a bolus of electrical stimulation to alleviate any acute pain or discomfort perceived by patient 102. If a characteristic of ECAP signals do correspond to a change in comfort or discomfort for the patient, IMD 106 may determine the change in ECAP that corresponded to the patient input identifying the change in comfort and adjust the target ECAP value or range used adjust stimulation.

In some examples, leads 108 may become dislodged, damaged, or caused to migrate within the tissue of patient 102, e.g., in response to a fall, a car accident, or the like. IMD 106 may, in response to sensor data and/or patient input indicating an occurrence of an event with a high change in acceleration, determine whether the signal characteristic(s) of the sensed ECAP signals after the occurrence of the event are within the target range(s) for target characteristic(s) of the current patient posture state. Based on a determination that the signal characteristic(s) are outside the target range(s), IMD 106 may determine a possibility of damage or migration of leads 108 and notify patient 102, e.g., via external programmer 120 and/or an external computing device (e.g., a smartwatch, a smartphone, a tablet, a laptop, or the like).

In the example techniques described in this disclosure, the control stimulation parameters and the target stimulation signal value (e.g., target range(s)of characteristic value(s)) of the stimulation signals may be initially set at the clinic but may be set and/or adjusted at home by patient 102. Once a target stimulation signal value (e.g., target range(s) of characteristic value(s)) are set, the example techniques allow for automatic adjustment of parameters of the stimulation pulses in order to maintain consistent volume of neural activation and consistent perception of therapy for patient 102 when the electrode-to-neuron distance changes. The ability to change the stimulation parameter value(s) may also allow the therapy to have long term efficacy, with the ability to keep the intensity of the stimulation (e.g., as indicated by the detected stimulation signals) consistent by comparing the measured characteristic value(s) of the stimulation signals to the target range(s) of characteristic values. IMDs 106 may perform these changes without intervention by a physician or patient 102.

FIG. 2 is a block diagram illustrating an example configuration of components of IMD 106, in accordance with one or more techniques of this disclosure. In the example shown in FIG. 2, IMD 106 includes stimulation generation circuitry 202, switch circuitry 204, sensing circuitry 206, communication circuitry 208, processing circuitry 210, storage device 212, sensor(s) 221, and power source 218. The components of IMD 106 may be secured within a housing 220.

In the example shown in FIG. 2, storage device 212 stores therapy stimulation programs 214 and patient scenarios 216 in separate memories within storage device 212 or in separate areas within storage device 212. Each stored therapy stimulation program of therapy stimulation programs 214 defines value(s) for a set of electrical stimulation parameters (e.g., a stimulation parameter set), such as a stimulation electrode combination, electrode polarity, current or voltage amplitude, pulse width, pulse rate, and pulse shape. In examples in which control pulses are provided to the patient without the need for informed pulses, a separate test stimulation program may not be needed. Instead, the test stimulation program for therapy that only includes control pulses may define the same control pulses as the corresponding therapy stimulation program for those control pulses.

Accordingly, in some examples, stimulation generation circuitry 202 generates electrical stimulation signals in accordance with the electrical stimulation parameters noted above. Other range(s) of stimulation parameter value(s) may also be useful and may depend on the target stimulation site within patient 102. While stimulation pulses are described, stimulation signals may be of any form, such as continuous-time signals (e.g., sine waves) or the like. Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 224A-D (collectively referred to herein as “electrodes 224”), 224A-D (collectively referred to herein as “electrodes 224”), or directed sensed signals from one or more of electrodes 222, 224 to sensing circuitry 206. In other examples, stimulation generation circuitry 202 and/or sensing circuitry 206 may include sensing circuitry to direct signals to and/or from one or more of electrodes 222, 224, which may or may not also include switch circuitry 204.

Sensing circuitry 206 monitors signals from any combination of electrodes 222, 224. In some examples, sensing circuitry 206 includes one or more amplifiers, filters, and analog-to-digital converters. Sensing circuitry 206 may be used to sense physiological signals, such as ECAP signals or other evoked signals. In some examples, sensing circuitry 206 detects electrical signals such as ECAPs from a particular combination of electrodes 222, 224. In some examples, the particular combination of electrodes for sensing ECAPs includes different electrodes than a set of electrodes 222, 224 used to deliver stimulation pulses. Alternatively, in other cases, the particular combination of electrodes used for sensing ECAPs includes at least one of the same electrodes as a set of electrodes used to deliver stimulation pulses to patient 102. Sensing circuitry 206 may provide signals to an analog-to-digital converter, for conversion into a digital signal for processing, analysis, storage, or output by processing circuitry 210.

Communication circuitry 208 supports wireless communication between IMD 106 and an external programmer (not shown in FIG. 2) or another computing device under the control of processing circuitry 210. Processing circuitry 210 of IMD 106 may receive, as updates to programs, value(s)for various stimulation parameters such as amplitude and electrode combination, from the external programmer via communication circuitry 208. Updates to the therapy stimulation programs 214 may be stored within storage device 212. Communication circuitry 208 in IMD 106, as well as communication circuits in other devices and systems described herein, such as the external programmer, may accomplish communication by radiofrequency (RF) communication techniques. In addition, communication circuitry 208 may communicate with an external medical device programmer (not shown in FIG. 2) via proximal inductive interaction of IMD 106 with the external programmer. The external programmer may be one example of external programmer 120 of FIG. 1. Accordingly, communication circuitry 208 may send information to the external programmer on a continuous basis, at periodic intervals, or upon request from IMD 106 or the external programmer.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 controls stimulation generation circuitry 202 to generate stimulation signals according to therapy stimulation programs 214 stored in storage device 212 to apply stimulation parameter value(s)specified by one or more of programs, such as amplitude, pulse width, pulse rate, and pulse shape of each of the stimulation signals.

In the example shown in FIG. 2, the set of electrodes 222 includes electrodes 222A, 222B, 222C, and 222D, and the set of electrodes 224 includes electrodes 224A, 224B, 224C, and 224D. In other examples, a single lead may include all eight electrodes 222 and 224 along a single axial length of the lead. Processing circuitry 210 also controls stimulation generation circuitry 202 to generate and apply the stimulation signals to selected combinations of electrodes 222, 224. In some examples, stimulation generation circuitry 202 includes a switch circuit (instead of, or in addition to, switch circuitry 204) that may couple stimulation signals to selected conductors within leads 108, which, in turn, deliver the stimulation signals across selected electrodes 222, 224. Such a switch circuit may be a switch array, switch matrix, multiplexer, or any other type of switching circuit configured to selectively couple stimulation energy to selected electrodes 222, 224 and to selectively sense bioelectrical neural signals of a spinal cord of the patient (not shown in FIG. 2) with selected electrodes 222, 224.

In other examples, however, stimulation generation circuitry 202 does not include a switch circuit and switch circuitry 204 does not interface between stimulation generation circuitry 202 and electrodes 222, 224. In these examples, stimulation generation circuitry 202 includes a plurality of pairs of voltage sources, current sources, voltage sinks, or current sinks connected to each of electrodes 222, 224 such that each pair of electrodes has a unique signal circuit. In other words, in these examples, each of electrodes 222, 224 is independently controlled via its own signal circuit (e.g., via a combination of a regulated voltage source and sink or regulated current source and sink), as opposed to switching signals between electrodes 222, 224.

Electrodes 222, 224 on respective leads 108 may be constructed of a variety of different designs. For example, one or both of leads 108 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 202, e.g., via switch circuitry 204 and/or switching circuitry of the stimulation generation circuitry 202, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 108. These and other constructions may be used to create a lead with a complex electrode geometry.

Although sensing circuitry 206 is incorporated into a common housing 220 with stimulation generation circuitry 202 and processing circuitry 210 in FIG. 2, in other examples, sensing circuitry 206 may be in a separate housing from IMD 200 and may communicate with processing circuitry 210 via wired or wireless communication techniques. In some examples, sensing circuitry 206 may be disposed in a separate IMD and may communicate with IMD 200 via wired or wireless communication techniques.

In some examples, one or more of electrodes 222 and 224 are suitable for sensing one or more ECAPs. For instance, electrodes 222 and 224 may sense the voltage amplitude of a portion of the ECAP signals, where the sensed voltage amplitude is a characteristic of the ECAP signal.

Storage device 212 may be configured to store information within IMD 106 during operation. Storage device 212 may include a computer-readable storage medium or computer-readable storage device. In some examples, storage device 212 includes one or more of a short-term memory or a long-term memory. Storage device 212 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210. As discussed above, storage device 212 is configured to store therapy stimulation programs 214 and patient scenarios 216.

Patient scenarios 216 may include a plurality of patient scenarios. Each patient scenario may be based on a different combination of a comfort level of patient 102 (e.g., a pain level value perceived by patient 102) a corresponding posture state patient 102, and signal characteristic value(s) of a corresponding sensed ECAP signal for the respective posture state. At least some patient scenarios may include, for a given posture, the corresponding ECAP signal for a desired comfort level. Such patient scenarios may be referred to herein as “baseline patient scenarios” and the corresponding ECAP signals may be referred to herein as “baseline ECAP signals”. Each baseline patient scenario may include, for the corresponding posture state and target characteristic(s) of the baseline ECAP signal for that posture state. The target characteristic(s) may be at least partially based on the prior sensed ECAP signals. The target characteristic(s) may include, but are not limited to, an amplitude of a portion of the signal, a nerve conduction velocity of the signal, an area under one or more peaks, frequency content, and/or maximum and/or minimum peak timing. For each target characteristic, patient scenarios 216 may store one or more target values. In some examples, patient scenarios 216 may store target value(s) and/or target range(s) of values for each target characteristic. Target range(s) of target characteristic may be a range of values for each posture state where the patient indicated (e.g., via patient input) a lack of pain or discomfort perceived by the patient. In some examples, the target range(s) for a target characteristic may be an amount of deviation from a single value for each posture state where the patient indicated the lack of perceived pain or discomfort for that specific posture state of the plurality of posture states. In some examples, each patient state and/or each target characteristic within patient scenarios 216 may correspond to one or more of therapy stimulation programs 214.

In some examples, stimulation generation circuitry 202 may be configured to deliver electrical stimulation therapy to patient 102. In some examples, the electrical stimulation therapy may include a plurality of informed pulses. Additionally, stimulation generation circuitry 202 may be configured to deliver a plurality of control pulses, where the plurality of control pulses is interleaved with at least some informed pulses of the plurality of informed pulses. Stimulation generation circuitry 202 may deliver the plurality of informed pulses and the plurality of control pulses to target tissue (e.g., spinal cord 104) of patient 102 via electrodes 222, 224 of leads 108. By delivering such informed pulses and control pulses, stimulation generation circuitry 202 may cause IMD 106 to sense stimulation signals that are indicative of the delivered pulses Additionally, or alternatively, stimulation generation circuitry 202 may deliver control pulses that evoke detectable responsive ECAPs in the target tissue, the responsive ECAPs propagating through the target tissue before arriving back at electrodes 222, 224. Stimulation signals or ECAPs caused by or elicited by informed pulses may not be detectable by IMD 106, or IMD 106 may not be configured to sense for ECAPs elicited by informed pulses. In some examples, a different combination of electrodes 222, 224 may sense responsive ECAPs and/or responsive stimulation signals than a combination of electrodes 222, 224 that delivers informed pulses and a combination of electrodes 222, 224 that delivers control pulses. Sensing circuitry 206 may be configured to detect the responsive ECAPs and/or the responsive stimulation signals via electrodes 222, 224 and leads 108. In other examples, stimulation generation circuitry 202 may be configured to deliver a plurality of control pulses, without any informed pulses, when control pulses also provide or contribute to a therapeutic effect for the patient.

In some examples, stimulation generation circuitry 202 may deliver multimodal stimulation (e.g., differential targeted multiplexed stimulation). Multimodal stimulation includes different stimulation pulses (e.g., a prime stimulation pulse and a base stimulation pulse) defined by different stimulation parameters such as different frequencies and different electrode combinations. Since different types of cells, such as glial cells and neurons, respond differently to electrical fields, it is then possible to differentially modulate the response of these cell populations with distinctly different electrical parameters. For example, the prime stimulation pulses can be delivered to affect glial cells (e.g., in spinal cord 104) and the base stimulation pulses can be delivered to affect neurons. Generally, the prime stimulation pulses are delivered at a higher pulse frequency than the base stimulation pulses, as described in greater detail herein.

Processing circuitry 210 may, in some cases, direct sensing circuitry 206 to continuously monitor for ECAPs signals. In other cases, processing circuitry 210 may direct sensing circuitry 206 to monitor for ECAPs signals periodically and/or based on signals from sensor(s) 221. Activating and deactivating sensing circuitry 206 to monitor for ECAP signals periodically rather than continuously monitoring for ECAP signals may, in some examples, extend a battery life of power source 218.

Processing circuitry 210 may determine, e.g., based on data from sensor(s) 221 a posture state of patient 102. For example, processing circuitry 210 may determine, based on accelerometer data from an accelerometer disposed within housing 220, a posture state of patient 102. In some examples, processing circuitry 210 may receive, via communications circuitry 208, patient input indicating the posture state of patient 102. IMD 106 may sense, e.g., via sensing circuitry 206, ECAP signals from tissue of patient 102 and determine signal characteristic(s) of the ECAP signals. In some examples, IMD 106 may receive signals corresponding to or representative of ECAP signals and determine the signal characteristic(s) of the ECAP signals based on the received signals. Processing circuitry 210 may receive, via communications circuitry 208, patient input indicating a comfort level perceived by patient 102 at a first time. The comfort level perceived by patient 102 may include the presence of and/or a level of pain experienced by patient 102 at the first time.

Processing circuitry 210 may generate patient scenarios 216 based at least in part on the posture state of patient 102, the sensed ECAP signals and/or the determined signal characteristic(s) of the sensed ECAP signals, and the patient input indicating comfort levels perceived by patient 102. For example, for each time period (e.g., the first time), processing circuitry 210 may determine the posture state of patient 102, the corresponding patient input indicating the comfort level perceived by patient 102, and corresponding sensed ECAP signals and/or determined signal characteristics. Processing circuitry 210 may compile, for each posture state, the corresponding comfort levels, sensed ECAP signals, and/or determined signal characteristic(s) for a plurality of time periods.

Processing circuitry 210 may determine, for each posture state, target value(s) for the determined signal characteristic(s) of ECAP signals, e.g., to achieve a desired comfort level perceived by patient 102. The desired comfort level perceived by patient 102 may be programmed and/or adjusted by the clinician, external programmer 120, and/or patient 102 and may include, a threshold level of pain, an absence of pain, an absence of discomfort, or the like. For example, processing circuitry 210 may determine, for a given posture state, a period of time when patient 102 did not experience discomfort. Processing circuitry 210 may then determine the signal characteristic(s) of ECAP signals during the period of time. Processing circuitry 210 may then select the determined signal characteristic(s) during the period of time as target characteristic(s) and the value(s) of the determined signal characteristic(s) as the target value(s) of the target characteristic(s). The target value(s) of the target characteristic(s) may be target value(s) of the target characteristic(s) of a baseline ECAP signal for patient 102 in the given posture state. The baseline for each posture state may include ECAP signal characteristic value(s) corresponding to patient 102 perceiving the desired comfort level in said posture state.

Patient 102 may experience an increased level of pain or discomfort over time (e.g., due to changes in a disease state of patient 102, due to the effects of therapeutic substances administer to patient 102. In such examples, processing circuitry 210 determines the posture state of patient 102 and compares signal characteristic(s) of currently sensed ECAP signals against the target signal characteristic(s) for the posture state, e.g., as stored in patient scenarios 216. Processing circuitry 210 may determine, based on the comparisons, whether patient 102 is perceiving an increased level of discomfort (e.g., as compared to the desired comfort level). For example, processing circuitry 210 may determine that, based on a determination that determined signal characteristic(s) of the currently sensed ECAP signals deviate outside the target range(s) of the respective target characteristic(s), that patient 102 is perceiving an increased level of discomfort. Processing circuitry 210 may then adjust parameters of the electrical stimulation therapy to treat the increased level of discomfort and cause stimulation generation circuitry 202 to deliver the adjusted therapy to patient 102. Processing circuitry 210 may repeat the process until processing circuitry 210 determines that the determined signal characteristic(s) of the currently sensed ECAP signals are within the target range(s) of the respective target characteristic(s) (e.g., patient 102 is perceiving the desired comfort level).

In some examples, patient 102 may change posture from a first posture state to a second posture state. In such examples, processing circuitry 210 may compare the determined signal characteristic(s) of the currently sensed ECAP signals against the target value(s) of the respective target characteristic(s) for the second posture state, e.g., as stored in patient scenarios 216. Processing circuitry 210 may then adjust the parameters of the electrical stimulation therapy and cause stimulation generation circuitry 202 to deliver the adjusted therapy to patient 102. Processing circuitry 210 may iteratively adjust the parameters of the electrical stimulation therapy until processing circuitry 210 determines that the determined signal characteristic(s) of the currently sensed ECAP signals are within the target range(s) of the respective target characteristic(s) for the second posture state.

In some examples, patient 102 may perceive an acute episode of pain or discomfort. Processing circuitry 210 may receive, via communications circuitry 208, patient input indicating a perception of the acute episode of pain or discomfort by patient 102. Processing circuitry 210 may determine whether any changes in the ECAP signals correspond to the patient input. Based on an identification of changes in the ECAP signals corresponding to the patient input, processing circuitry 210 may adjust parameters of the electrical stimulation therapy. In some examples, processing circuitry 210 may adjust (e.g., temporarily) or maintain the parameters of the electrical stimulation therapy if processing circuitry 210 cannot identify any corresponding changes in the ECAP signals. Temporary adjustment or maintenance of the parameters may reduce power consumption by components of IMD 106 and/or reduce patient discomfort when the acute episode subsides. In some examples, one or more therapy stimulation programs 214 may be pre-selected (e.g., by the clinician) to treat acute episodes of pain or discomfort. In such examples, processing circuitry 210 may select a pre-selected therapy stimulation program 214 (e.g., based on posture state of patient 102, based on signal characteristic(s) of the ECAP signals, based on a level of discomfort perceived by patient 102) and cause stimulation generation circuitry 202 to deliver a bolus of electrical stimulation therapy in accordance with the selected therapy stimulation program 214, e.g., until patient 102 no longer perceives the pain or discomfort.

In some examples, high-energy impacts to patient 102 (e.g., falls, car accidents, or the like) may damage leads 108 and/or cause leads 108 to migrate, e.g., away from tissue adjacent spinal cord 104. In such examples, the changes in position and/or integrity of leads 108 may alter the electrical signals delivered by leads 108 to nerves and, by extension, the ECAP signals sensed along the nerves.

Processing circuitry 210 may determine the occurrence of an impact event, e.g., based on rapid changes in acceleration detected by sensor(s) 221. Based on the determination, processing circuitry 210 may compare the determined signal characteristic(s) of the currently sensed ECAP signals against the target value(s)of the respective target characteristic(s) for the current posture state of patient 102. Based on a determination that the determined signal characteristic(s) exceed the target range(s), processing circuitry 210 may determine a possibility of damage, migration, and/or other changes to lead 108 as a result of the impact event. Processing circuitry 210 may then cause communications circuitry 208 to notify the clinician, the external programmer (e.g., external programmer 120), and/or patient 102 of possible damage and/or migration due to the impact event. In some examples, processing circuitry 210 may, based on the determination of the possibility of damage and/or migration of leads 108, processing circuitry 210 may then initiate a comprehensive diagnostic process to evaluate the integrity of leads 108.

Power source 218 is configured to deliver operating power to the components of IMD 106. Power source 218 may include a battery and a power generation circuit to produce the operating power. In some examples, the battery is rechargeable to allow extended operation. In some examples, recharging is accomplished through proximal inductive interaction between an external charger and an inductive charging coil within IMD 106. Power source 218 may include any one or more of a plurality of different battery types, such as nickel cadmium batteries and lithium ion batteries.

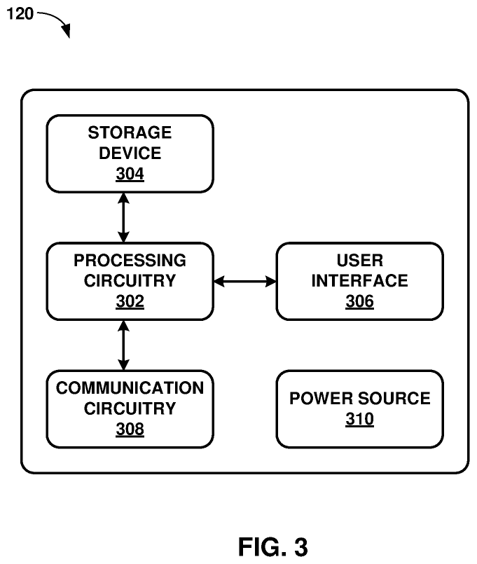

FIG. 3 is a block diagram illustrating an example configuration of components of external programmer 120, in accordance with one or more techniques of this disclosure. Although external programmer 120 may generally be described as a hand-held device, external programmer 120 may be a larger portable device or a more stationary device. In addition, in other examples, external programmer 120 may be included as part of an external charging device or include the functionality of an external charging device. As illustrated in FIG. 3, external programmer 120 may include processing circuitry 302, storage device 304, user interface (UI) 306, communications circuitry 358, and power source 310. Storage device 304 may store instructions that, when executed by processing circuitry 302, cause processing circuitry 302 and external programmer 120 to provide the functionality ascribed to external programmer 120 throughout this disclosure. Each of these components, circuitry, or modules, may include electrical circuitry that is configured to perform some, or all of the functionality described herein. For example, processing circuitry 302 may include processing circuitry configured to perform the processes discussed with respect to processing circuitry 302.

In general, external programmer 120 includes any suitable arrangement of hardware, alone or in combination with software and/or firmware, to perform the techniques attributed to external programmer 120, and processing circuitry 302, user interface 306, and communications circuitry 308 of external programmer 120. In various examples, external programmer 120 may include one or more processors, such as one or more microprocessors, DSPs, ASICs, FPGAs, or any other equivalent integrated or discrete logic circuitry, as well as any combinations of such components. External programmer 120 also, in various examples, may include a storage device 304, such as RAM, ROM, PROM, EPROM, EEPROM, flash memory, a hard disk, a CD-ROM, including executable instructions for causing the one or more processors to perform the actions attributed to them. Moreover, although processing circuitry 302 and communications circuitry 308 are described as separate modules, in some examples, processing circuitry 302 and communications circuitry 308 are functionally integrated. In some examples, processing circuitry 302 and communications circuitry 308 correspond to individual hardware units, such as ASICs, DSPs, FPGAs, or other hardware units.

Storage device 304 (e.g., a storage device) may store instructions that, when executed by processing circuitry 302, cause processing circuitry 302 and external programmer 120 to provide the functionality ascribed to external programmer 120 throughout this disclosure. For example, storage device 304 may include instructions that cause processing circuitry 302 to instruct IMDs 106 to deliver stimulation pulses to patient 102 in accordance with one or more therapy stimulation programs 214 (not pictured) stored in one or more of IMDs 106 or external programmer 120. In some examples, storage device 304 may store information corresponding prior sensed ECAP signals and/or patient scenarios 216 (not pictured). For example, storage device 305 may store characteristic values, and corresponding posture states (e.g., corresponding sensor data from sensor(s) 221 of IMD 106, corresponding patient in put) for one or more ECAP signals sensed by IMD 106. Some of patient scenarios 216 may include different posture states of patient 102 and corresponding target value(s) of target characteristic(s) of a baseline ECAP signal of patient 102 for the particular posture state.

Storage device 304 may include a plurality of therapy stimulations programs, where each program includes a parameter set that defines stimulation pulses, such as prime stimulation pulses and/or base stimulation pulses. Storage device 304 may also store data received from a medical device (e.g., IMD 106). The data may include, but is not limited to, sensed stimulation signals, sensed ECAP signals, and stimulation parameters (e.g., timing parameters) for a stimulation pulse configured to be delivered by the medical device. For example, storage device 304 may store stimulation signals and/or ECAP related data recorded at a sensing module of the medical device, and storage device 304 may also store data from one or more sensors of the medical device.

User interface 306 may include a button or keypad, lights, a speaker for voice commands, a display, such as a liquid crystal (LCD), light-emitting diode (LED), or organic light-emitting diode (OLED). In some examples the display includes a touch screen. User interface 306 may be configured to display any information related to the delivery of electrical stimulation, identified patient behaviors, sensed patient parameter values, patient behavior criteria, or any other such information. User interface 306 may also receive patient input via user interface 306. The patient input may be, for example, in the form of pressing a button on a keypad or selecting an icon from a touch screen. The patient input may indicate a comfort level perceived by patient 102 at a particular point in time (e.g., a comfort level patient 102 is currently perceiving). The patient input may also include indicate of a current posture state of patient 102 (e.g., standing, sitting, crouching, lying down, etc.). In some examples, patient 102 may enter, into user interface 306, patient input indicating perception of a suddenly onsetting pain or discomfort (i.e., acute pain).

Communications circuitry 308 may support wireless communication between IMD 106 and external programmer 120 under the control of processing circuitry 302. Communications circuitry 308 may also be configured to communicate with another computing device via wireless communication techniques, or direct communication through a wired connection. In some examples, communications circuitry 308 provides wireless communication via an RF or proximal inductive medium. Another computing device may include a medical device (e.g., a wearable medical device, an IMD) configured to communicate with IMD 106 and external programmer 120. In such examples, IMD 106 may communicate with external programmer 120 via the medical device and may not directly communicate with external programmer 120. In some examples, communications circuitry 308 includes an antenna, which may take on a variety of forms, such as an internal or external antenna.

Examples of local wireless communication techniques that may be employed to facilitate communication between external programmer 120 and IMD 106 include RF communication according to the 802.11 or Bluetooth® specification sets or other standard or proprietary telemetry protocols. In this manner, other external devices may be capable of communicating with external programmer 120 without needing to establish a secure wireless connection. As described herein, communications circuitry 308 may be configured to transmit a spatial electrode movement pattern or other stimulation parameter value(s)to IMD 106 for delivery of electrical stimulation therapy.

In some examples, stimulation parameters of therapy stimulation programs 214 are transmitted to IMD 106 for delivery to a patient (e.g., patient 102 of FIG. 1). In other examples, the therapy may include medication, activities, or other instructions that patient 102 may perform themselves or a caregiver may perform for patient 102. In some examples, external programmer 120 provides visual, audible, and/or tactile notifications that indicate there are new instructions. External programmer 120 requires receiving patient input acknowledging that the instructions have been completed in some examples.

According to the techniques of the disclosure, user interface 306 of external programmer 120 receives an indication from a clinician instructing a processor of the medical device (e.g., processing circuitry 210 of IMD 106) to update one or more of therapy stimulation programs or to generate and/or update patient scenarios 216 stored in the medical device. Updating therapy stimulation programs may include changing one or more parameters (e.g., timing parameters) of the stimulation pulses delivered by IMD 106 according to the programs, such as amplitude, pulse width, frequency, timing, pulse shape, and/or type of pulse (e.g., informed pulses, control pulses, prime pulses, base pulses). Updating patient scenarios may include updating target value(s) and/or target range(s) for target characteristic(s) of ECAP signals for one or more posture states stored in patient scenarios 216. User interface 306 may also receive instructions from the clinician commanding any electrical stimulation, including control pulses and/or informed pulses to commence or to cease.

In some examples, based on instructions received via user interface 306, processing circuitry 302 may transmit patient input information to a medical device (e.g., to IMD 106) via communications circuitry 308. IMD 106 may then perform the functions previously described herein based at least in part on the patient inputs received from communications circuitry 308. For example, IMD 106 may generate and/or update a patient scenario (e.g., of patient scenarios 216) defining various target values and/or ranges for target characteristics of baseline ECAP signals for various posture states. In some examples, IMD 106 may determine whether to adjust parameters of an electrical stimulation therapy based on reception of patient input indicating perception of an acute episode of pain or discomfort by patient 102. In some examples, IMD 106 may adjust the target values of baseline ECAP signals and/or one or more therapy stimulation programs based on the received patient input. In some examples, processing circuitry 302 may cause communications circuitry 308 to transmit instructions on how to adjust the target values and/or the one or more therapy stimulation programs. In some examples, processing circuitry 210 of IMD 106 may perform the adjustments automatically in a closed-loop process (e.g., based on sensed ECAP signals and/or the received patient input). In some examples, where patient 102 reports perceiving an acute episode of pain or discomfort and IMD 106 cannot correspond the acute episode to changes in the sensed ECAP signals, processing circuitry 302 may cause communications circuitry 308 to transmit instruction for a predetermined electrical stimulation therapy (e.g., stimulation parameters for the predetermined electrical stimulation therapy) and cause IMD 106 to deliver a bolus of stimulation based on the predetermined electrical stimulation therapy, e.g., to alleviate the acute pain or discomfort experienced by patient 102.

Power source 310 is configured to deliver operating power to the components of external programmer 120. Power source 310 may include a battery and a power generation circuit to produce the operating power. In some examples, the battery is rechargeable to allow extended operation. Recharging may be accomplished by electrically coupling power source 310 to a cradle or plug that is connected to an alternating current (AC) outlet. In addition, recharging may be accomplished through proximal inductive interaction between an external charger and an inductive charging coil within external programmer 120. In other examples, traditional batteries (e.g., nickel cadmium or lithium ion batteries) may be used. In addition, external programmer 120 may be directly coupled to an alternating current outlet to operate.

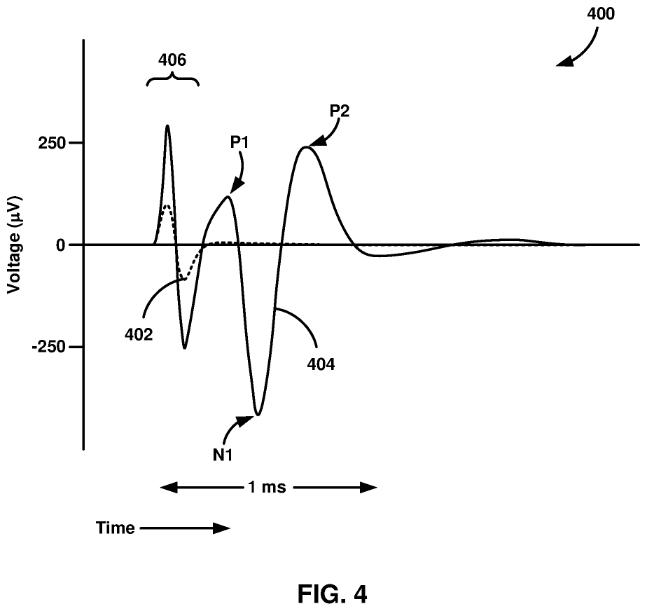

FIG. 4 is a graph 400 of example evoked compound action potentials (ECAPs) sensed for respective stimulation pulses, in accordance with one or more techniques of this disclosure. As shown in FIG. 4, graph 400 shows example ECAP signal 402 (dotted line) and ECAP signal 404 (solid line). In some examples, each of ECAP signals 402 and 404 are sensed from stimulation pulses (e.g., a control pulse, a base pulse) that were delivered from a guarded cathode, where the stimulation pulses are bi-phasic pulses including an interphase interval between each positive and negative phase of the pulse. In some such examples, the guarded cathode includes stimulation electrodes located at the end of an 8-electrode lead (e.g., leads 108 of FIG. 1) while two sensing electrodes are provided at the other end of the 8-electrode lead. ECAP signal 402 illustrates the voltage amplitude sensed as a result from a sub-detection threshold stimulation pulse, or a stimulation pulse which results in no detectable ECAP. It is noted that monophasic, tri-phasic, or pulses with another quantity of phases may be in other examples.

Peaks 406 of ECAP signal 402 are detected and represent stimulation signals of the delivered stimulation pulse. However, no propagating signal is detected after the stimulation signal in ECAP signal 402 because the stimulation pulse had an intensity (e.g., an amplitude and/or pulse width) that was “subthreshold” or below a detection threshold (e.g., a sub-detection threshold) and/or below a propagation threshold (e.g., a sub-propagation threshold).

In contrast to ECAP signal 402, ECAP signal 404 represents the voltage amplitude detected from a supra-detection stimulation threshold stimulation pulse. Peaks 406 of ECAP signal 404 are detected and represent stimulation signals of the delivered stimulation pulse. After peaks 406, ECAP signal 404 also includes peaks P1, N1, and P2, which are three typical peaks representative of propagating action potentials from an ECAP. The example duration of the stimulation signal and peaks P1, N1, and P2 is approximately 1 millisecond (ms).

When detecting the ECAP of ECAP signal 404, different characteristic(s)may be identified. For example, the characteristic of the ECAP may be the amplitude between N1 and P2. This N1-P2 amplitude may be easily detectable even if the stimulation signal impinges on P1, a relatively large signal, and the N1-P2 amplitude may be minimally affected by electronic drift in the signal. In other examples, the characteristic of the ECAP used to control subsequent stimulation pulses (e.g., control pulses and/or informed pulses) may be an amplitude of P1, N1, or P2 with respect to neutral or zero voltage. In some examples, the characteristic of the ECAP used to control subsequent stimulation pulses is a sum of two or more of peaks P1, N1, or P2. In other examples, the characteristic of ECAP signal 404 may be the area under one or more of peaks P1, N1, and/or P2. In other examples, the characteristic of the ECAP may be a ratio of one of peaks P1, N1, or P2 to another one of the peaks. In some examples, the characteristic of the ECAP is a slope between two points in the ECAP signal, such as the slope between N1 and P2. In other examples, the characteristic of the ECAP may be the time between two points of the ECAP, such as the time between N1 and P2.

The time between when the stimulation pulse is delivered and a point in the ECAP signal may be referred to as a latency of the ECAP and may indicate the types of fibers being captured by the stimulation pulse (e.g., a control pulse). In this manner, the latency may be determined from multiple points on the sensed ECAP signal. ECAP signals with lower latency (i.e., smaller latency values) indicate a higher percentage of nerve fibers that have faster propagation of signals, whereas ECAP signals with higher latency (i.e., larger latency values) indicate a higher percentage of nerve fibers that have slower propagation of signals. Latency may also refer to the time between an electrical feature is detected at one electrode and then detected again at a different electrode. This time, or latency, is inversely proportional to the conduction velocity of the nerve fibers. Other characteristic(s)of the ECAP signal may be used in other examples. In some examples, a change in pain may be reflected by different fiber activations which may manifest in a change in latency.

The amplitude of the ECAP signal may increase with increased amplitude of the stimulation pulse, as long as the pulse amplitude is greater than threshold such that nerves depolarize and propagate the signal and there are additional fibers to activate with the increased amplitude of the stimulation pulse. The target ECAP characteristic (e.g., the target ECAP amplitude) may be determined from the ECAP signal detected from a stimulation pulse (or a control pulse) when informed pulses are determined to deliver effective therapy to patient 102. The ECAP signal thus is representative of the distance between the stimulation electrodes and the nerves appropriate for the stimulation parameter value(s)of the informed pulses delivered at that time. Additionally, the ECAP signal may be correlated with patient posture states and perceptions of comfort level by patient 102. In such examples, the ECAP signals may be used to represent a comfort level perceived by patient 102 in a given posture state (e.g., when patient 102 is standing, when patient 102 is sitting).

When patient 102 is remaining in a posture state, the position of electrodes of IMD 106 may remain constant relative to target tissue of patient 102. Nonetheless, the ECAP signals detected by IMD 106 may change over time, e.g., due to progression in a disease state of patient 102, effects of therapeutic substances. Additionally, the ECAP signals detected by IMD 106 may change when patient 102 changes posture states. IMD 106 may to use the detected changes to the measured ECAP characteristic value to change stimulation parameter value(s), e.g., to cause patient 102 to perceive a target comfort level. In some examples, IMD 106 may update target value(s) and/or target range(s) for target ECAP characteristics of baseline ECAP signals based on the detected changes in the measured ECAP characteristics.

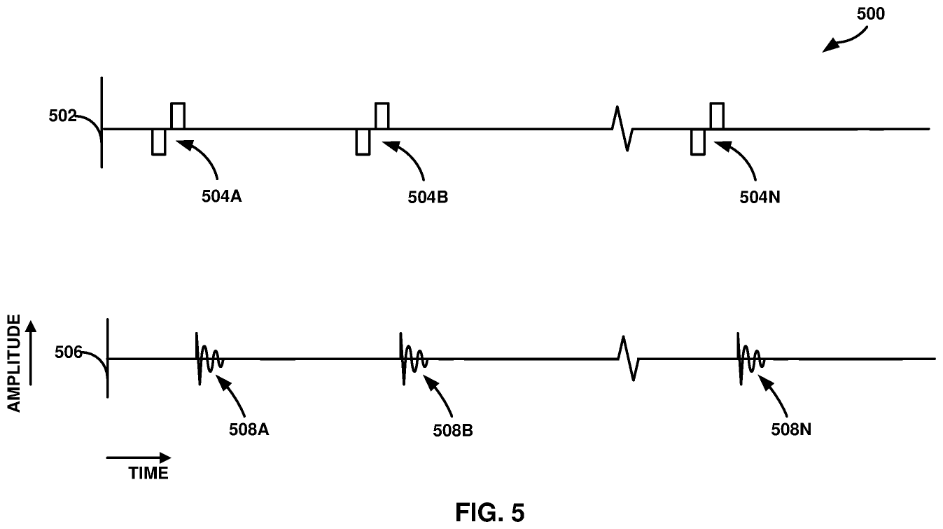

FIG. 5 is a timing diagram 500 illustrating an example of electrical stimulation pulses, respective stimulation signals, and respective sensed ECAPs, in accordance with one or more techniques of this disclosure. For convenience, FIG. 5 is described with reference to IMD 106 of FIG. 2. As illustrated, timing diagram 5 includes first channel 502, a plurality of stimulation pulses 504A-504N (collectively “stimulation pulses 504”), second channel 506, and a plurality of respective ECAPs 508A-508N (collectively “ECAPs 508”). In some examples, stimulation pulses 504 may represent control pulses which are configured to elicit ECAPs 508 that are detectible by IMD 200, but this is not required. In some examples, stimulation pulses 504 may represent base pulses. Stimulation pulses 504 may represent any type of pulse that is deliverable by IMD 200. In the example of FIG. 5, IMD 200 can deliver therapy with control pulses instead of, or without, informed pulses.

First channel 502 is a time/voltage (and/or current) graph indicating the voltage (or current) of at least one electrode of electrodes 222, 224. In one example, the stimulation electrodes of first channel 502 may be located on the opposite side of the lead as the sensing electrodes of second channel 506. Stimulation pulses 504 may be electrical pulses delivered to the spinal cord of the patient by at least one of electrodes 222, 224, and may be shown with a negative phase and a positive phase separated by an interphase interval. For example, a stimulation pulse 504 may have a negative voltage for the same amount of time and amplitude that it has a positive voltage. It is noted that the negative voltage phase may be before or after the positive voltage phase. Stimulation pulses 504 may be delivered according to therapy stimulation programs 214 stored in storage device 212 of IMD 106. Therapy stimulation programs 214 may be updated according to patient input via an external programmer (e.g., external programmer 120) Each of stimulation pulses 504 may have a pulse width of less than approximately 300 microseconds (e.g., the total time of the positive phase, the negative phase, and the interphase interval is less than 300 microseconds). In some examples, each of stimulation pulses 504 may have a pulse width of approximately 100 □S for each phase of the bi-phasic pulse. As illustrated in FIG. 5, stimulation pulses 504 may be delivered via channel 502. Delivery of stimulation pulses 504 may be delivered by leads 108 in a guarded cathode electrode combination. For example, if leads 108 are linear 8-electrode leads, a guarded cathode combination is a central cathodic electrode with anodic electrodes immediately adjacent to the cathodic electrode.

Second channel 506 is a time/voltage (and/or current) graph indicating the voltage (or current) of at least one electrode of electrodes 222, 224. In one example, the electrodes of second channel 506 may be located on the opposite side of the lead as the electrodes of first channel 502. ECAPs 508 may be sensed at electrodes 222, 224 from the spinal cord of the patient in response to stimulation pulses 504. ECAPs 508 are electrical signals which may propagate along a nerve away from the origination of stimulation pulses 504. In one example, ECAPs 508 are sensed by different electrodes than the electrodes used to deliver stimulation pulses 504. As illustrated in FIG. 5, ECAPs 508 may be recorded on second channel 506.

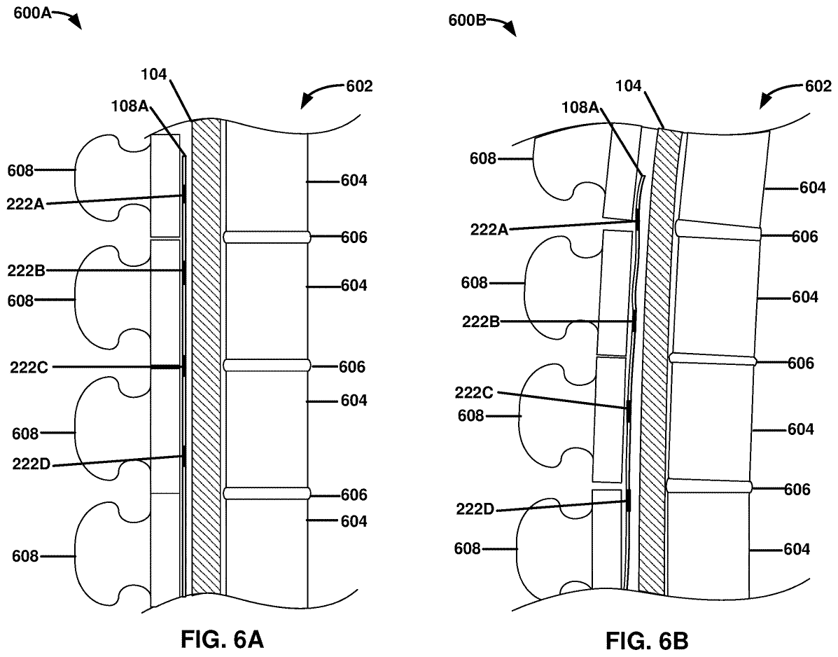

FIG. 6A is a conceptual diagram illustrating a spine 602 of a patient 102 in a first posture 600A, in accordance with the one or more techniques of this disclosure. FIG. 6B is a conceptual diagram illustrating spine 602 of FIG. 6A in a second posture 600B, in accordance with the one or more techniques of this disclosure. Spine 602 includes a plurality of vertebrae 604 extending down the back of patient 102. Adjacent vertebrae 604 are separated by intervertebral disks 606. Vertebrae 604 defines a channel extending along spine 602. Each of vertebrae 604 defines a spinal process 608 on the dorsal side of spine 602 and at least partially defining the channel. Spinal cord 104 is disposed within the channel that runs through vertebrae 604 of spine 602. Vertebrae 604 may include spinous processes 608 extending away from spinal cord 104.

One or more leads 108 may be implanted in the epidural space adjacent to spinal cord 104 or spine 602. In some examples, as illustrated in FIGS. 6A and 6B, one or more leads 108 (e.g., lead 108A) may be disposed within a channel defined by vertebrae 604 and adjacent to spinal cord 104. As illustrated, lead 108A includes electrodes 222 disposed at different longitudinal positions along the length of at least a distal portion of lead 108A. Each of electrodes 222 may be configured to deliver electrical stimulation signals to and/or sense electrical signals (e.g., ECAP) from nerves and/or fiber bundles a part of spinal cord 104 and/or extending out from spinal cord 104. Each of electrodes 222 may deliver the electrical stimulation signals and/or sense electrical signals from a different region along the length of spinal cord 104. In some examples, leads 108 may be placed laterally or anterior-laterally with respect to spinal cord 104 in order to sense pain signals that may travel through the lateral part of the spinal cord and/or the dorsal root. In some examples, a sensing-specific lead may be placed at this lateral position, and the stimulation leads may be placed more medial with respect to spinal cord 104. This sensing lead placed closer to potential sources of pain signals may improve pain sensing using ECAPs or other signals as described herein.

As illustrated in FIGS. 6A and 6B, when patient 102 shifts between posture states, e.g., from first posture 600A to second posture 600B, spine 602 and spinal cord 104 may shift accordingly. The movement of spine 602 and spinal cord 104 may cause lead 108 to move within the epidural space in spine 602 and dispose electrodes 222 near different nerves and/or fiber bundles connected to spinal cord 104. In some examples, lead 108 may remain relatively static as spine 602 and spinal cord 104 moves, thereby disposing electrodes 222 near different nerves and/or fiber bundles. The change in connections between electrodes 222 and nerves of patient 102 may cause IMD 106 to detect ECAP signals having different signal characteristic values, e.g., due to differences in physiology of the nerves, the proximity of electrodes 222 to the nerves and/or to spinal cord 104, etc. In some examples, one or more of electrodes 222 may retain near the same nerves and/or fiber bundles despite changes in the posture state of patient 102. In such examples, IMD 106 may detect ECAP signals having same signal characteristic values for the different posture states.

Within each posture state, the effects of the location of electrodes 222 on the sensed ECAP signal characteristics may remain substantially constant. Therefore, for each posture state system 100 may correlate particular signal characteristic values with different comfort levels perceived by patient 102. Based on the association of signal characteristic values with different comfort levels, system 100 may determine a baseline ECAP signal for each posture state and optimal target value(s) and/or target ranges for each target characteristic of each baseline ECAP signal. System 100 may then generate patient scenarios (e.g., patient scenarios 216), wherein, for each posture state (e.g., for first posture 600A, for second posture 600B), system 100 stores target value(s) and/or target range(s) for target characteristic(s) of sensed ECAP signals.

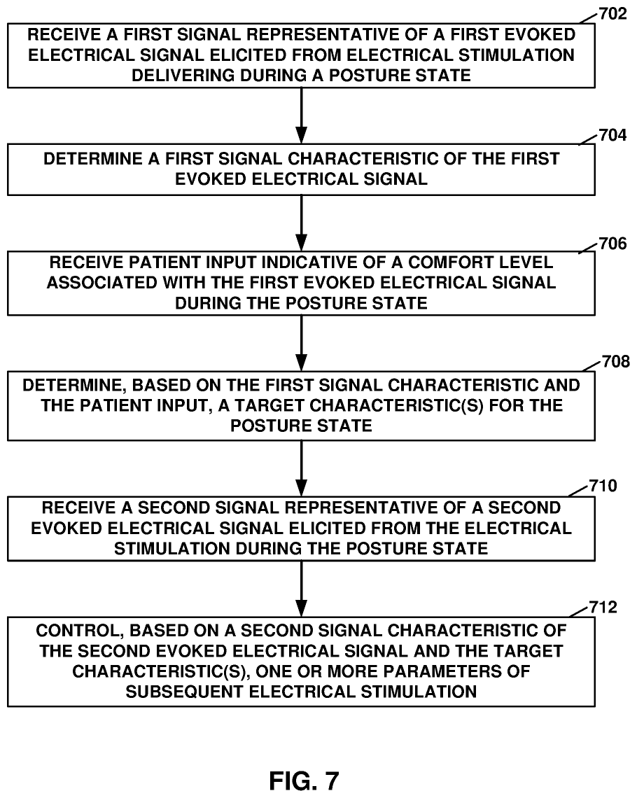

FIG. 7 is a flow diagram illustrating an example process for delivering stimulation therapy to a patient based on a sensed evoked signal and a patient posture state, in accordance with one or more techniques of this disclosure. While FIG. 7 describes system 100 with reference to one IMD (e.g., IMD 106), system 100 may include two or more IMDs. In addition, while the example process of FIG. 7 is primarily described as being performed by IMD 106 of system 100, the example process described below may be performed by another medical device (e.g., a wearable medical device, another IMD), by an external programmer of system 100 (e.g., external programmer 120), and/or other computing devices, systems, and/or cloud computing environments in communication with any components of system 100.

IMD 106 may receive a first signal representative of a first evoked electrical signal elicited from electrical stimulation delivering during a posture state (702). IMD 106 may receive the first signal from one or more sensors within IMD 106 such as from sensor(s) 221. In some examples, system 100 may receive the first signal from electrodes (e.g., electrodes 222, 224) disposed on implantable leads (e.g., leads 108) connected to IMD 106. IMD 106 may process the received first signal to identify the first evoked electrical signal. The first evoked electrical signal may include a first ECAP signal, although in some examples, the first evoked electrical signal may include other action potential signals.

IMD 106 may determine the posture state of patient 102 based on sensor data from sensor(s) 221 of IMD 106. For example, IMD 106 may determine an orientation of IMD 106 based on the sensor data from sensor(s) 221 and determine the posture state of patient 102 (e.g., standing, crouching, slouching, sitting, lying down) based on the determined orientation of IMD 106. Sensor(s) 221 may include, but are not limited to, accelerometers, gyroscopes, and/or inertial measurement units (IMUs). In some examples, System 100 may determine, or confirm, the posture state based on patient input received by external programmer 120 and/or based on external sensor data (e.g., from a camera connected to system 100). IMD 106 may receive the determined posture state via external programmer 120. In some examples, the sensor(s) may be disposed outside of IMD 106 and within, on, or otherwise associated with patient 102. For example, the sensor(s) may be disposed on a computer device worn or used by patient 102 (e.g., a smartwatch, a smartphone). The sensor(s) may communicate with IMD 106 and/or external programmer 120 and may transmit the sensor data to IMD 106.

IMD 106 may determine a first signal characteristic of the first evoked electrical signal (704). Signal characteristics may include, but are not limited to, an amplitude of a portion of the first evoked electrical signal, a nerve conduction velocity of the signal, an area under one or more peaks of the first evoked electrical signal, frequency content, and/or maximum and/or minimum peak timing of the first evoked electrical signal. IMD 106 may determine a value for one or more first signal characteristics based on the first evoked electrical signal.

IMD 106 may receive patient input indicative of a comfort level associated with the first evoked electrical signal during the posture state (706). IMD 106 may receive, e.g., via user interface 306 of external programmer 120, patient input indicating comfort levels perceived by patient 102 at different times. IMD 106 may then correlate the comfort levels in time with the first evoked electrical signal to determine a comfort level associated with the first evoked electrical signal. The comfort level perceived by patient 102 may include the presence, or lack thereof, of pain or discomfort perceived by patient 102, a type of pain or discomfort perceived by patient 102, a location of pain or discomfort perceived by patient 102, and/or a level of pain or discomfort perceived by patient 102. For example, the patient input may indicate that patient 102 is perceiving pain, that patient 102 is perceiving a sharp pain (e.g., versus a dull pain, a throbbing pain, etc.), that patient 102 is perceiving the sharp pain in their lower back, and/or that patient 102 is perceiving the sharp pain to be moderate (e.g., on a pain scale).

In some examples, in addition to the first evoked electrical signal, IMD 106 may detect a plurality of evoked electrical signals during the posture and associate comfort levels with each evoked electrical signal. The plurality of evoked electrical signals may be associated with varying comfort levels. IMD 106 may determine, based on the plurality of evoked electrical signals and the associated comfort levels, relationships between one or more signal characteristics of the evoked electrical signals and the comfort level perceived by patient 102.

IMD 106 may determine, based on the first signal characteristic and the patient input, a target characteristic for the posture state (708). IMD 106 may determine, based on the first signal characteristic and the patient input, a desired comfort level for patient 102 in the posture state. The desired comfort level may include the absence of pain and/or discomfort, or may include the presence of pain and/or discomfort below a threshold level of severity. The desired comfort level may be predetermined by a clinician or by patient 102 and may be transmitted to IMD 106 via external programmer 120. For each posture state, IMD 106 may identify the desired comfort level and determine target value(s) and target range(s) for one or more signal characteristics in evoked electrical signals that may correlate to the desired comfort level. The one or more signal characteristics may also be referred to as target characteristic(s) for the posture state.

In some examples, IMD 106 may determine that the first evoked electrical signal corresponds to a desired comfort level and store the target characteristic values of the first evoked electrical signal as the target value(s) and/or target range(s). In some examples, IMD 106 may determine a deviation between the comfort level corresponding to the first evoked electrical signal and the desired comfort level and calculate the target value(s) and/or target range(s) of the target characteristic(s) based on the level of deviation from the desired comfort level and the target characteristic values of the first evoked electrical signal. In some examples, IMD 106 may determine the target value(s) and/or target range(s) for the target characteristic(s) based on the plurality of evoked electrical signals and the corresponding comfort levels.

IMD 106 may determine the target value(s) and/or target range(s) for each target characteristic of one or more target characteristics and for the target characteristics of each posture state of one or more posture state. In some examples, different postures states may use different target characteristics. In some examples, target values for a same target characteristic for different posture state may be the same, e.g., due to electrodes of leads 108 of IMD 106 sensing evoked electrical signals from the same nerves and/or the same fiber bundles of nerves. The target value(s) and/or target range(s) for target characteristic(s) may be stored in IMD 106 (e.g., in patient scenarios 216 of storage device 212) and/or transmitted to external programmer 120. In some examples, the target value(s) and/or target range(s) for target characteristic(s) may define a baseline evoked electrical signal for the posture state.

In some examples, IMD 106 may adjust the electrical stimulation therapy based at least in part on the target value(s) and/or target range(s) for the target characteristic(s) of the baseline evoked electrical signal. For example, IMD 106 may determine a minimum stimulation amplitude capable of generating the baseline evoked electrical signal (e.g., a minimum amplitude configured to cause patient 102 to perceive the desired comfort level, a minimum amplitude configured to reduce or eliminate pain or discomfort perceived by patient 102). IMD 106 may then determine parameter value(s) for the electrical stimulation therapy based on the determined minimum stimulation amplitude and deliver electrical stimulation to patient 102 based on the determined parameter value(s). Delivery of electrical stimulation having the minimum stimulation amplitude may reduce excess power consumption and increase an operational lifespan of IMD 106, thereby necessitating fewer medical operations to replace IMD 106 and/or replace a power source of IMD 106. The increase in operation lifespan of IMD 106 may also increase an amount of time between charging sessions of IMD 106.

IMD 106 may receive a second signal representative of a second evoked electrical signal elicited from the electrical stimulation during the posture state (710). The second signal may be later in time than the first signal and may be in response to a different pulse of electrical stimulation. IMD 106 may determine the second evoked electrical signal based on the second signal. IMD 106 may determine for the second evoked electrical signal, values for one or more signal characteristics. The one or more signal characteristics of the second evoked electrical signal may be of same types as target characteristic(s) for the posture state.

IMD 106 may control, based on a second signal characteristic of the second evoked electrical signal and the target characteristic, one or more parameters of subsequent electrical stimulation (712). IMD 106 may compare a second signal characteristic of the one or more signal characteristics of the second evoked electrical signal against a target characteristic, e.g., to determine a deviation between the first evoked electrical signal and the second evoked electrical signal. Deviations may be caused by movement and/or damage to leads 108, progression of a disease state, effects of therapeutic substances, changes in physiology of patient 102, or the like. Based on the deviations, the electrical stimulation may no longer cause patient 102 to perceive the desired comfort level. If IMD 106 determines that the deviations are outside of a target range, IMD 106 may control one or more parameters of the electrical stimulation therapy (e.g., electrode combination, voltage or current amplitude, pulse width, pulse frequency) to cause patient 102 to perceive the desired comfort level.

Changing the one or more parameters may also change one or more aspects of the evoked electrical signals. IMD 106 may iteratively adjust the one or more parameters until IMD 106 determines that the deviation of the values of the second signal characteristic of the evoked electrical signals is within a target range of the target values of the target characteristic. IMD 106 may determine that the deviation between the second signal characteristic and the target characteristic exceeds a threshold amount and adjust the one or more parameters based on the determination. In some examples, IMD 106 may determine the deviation between the first evoked electrical signal or the baseline electrical signal and the second evoked electrical signal based on the comparison of two or more signal characteristics against the corresponding target characteristics.

In some examples, changes in patient physiology and/or progression in a patient disease state may affect nerve propagation capabilities and/or impact the characteristics of evoked electrical signals generated by the nerves in response to an electrical stimulation signal. The changes may be reflected in the differences between the second signal characteristic and the target characteristic. In some examples, IMD 106 may adjust the target characteristic to account for such changes. IMD 106 may adjust target values and/or target ranges of the target characteristic, e.g., to match the value of the second signal characteristic, to match a median or average value of a plurality of signal characteristics including the second signal characteristic, or otherwise adjust the target values and/or target ranges based at least in part on the second signal characteristic.

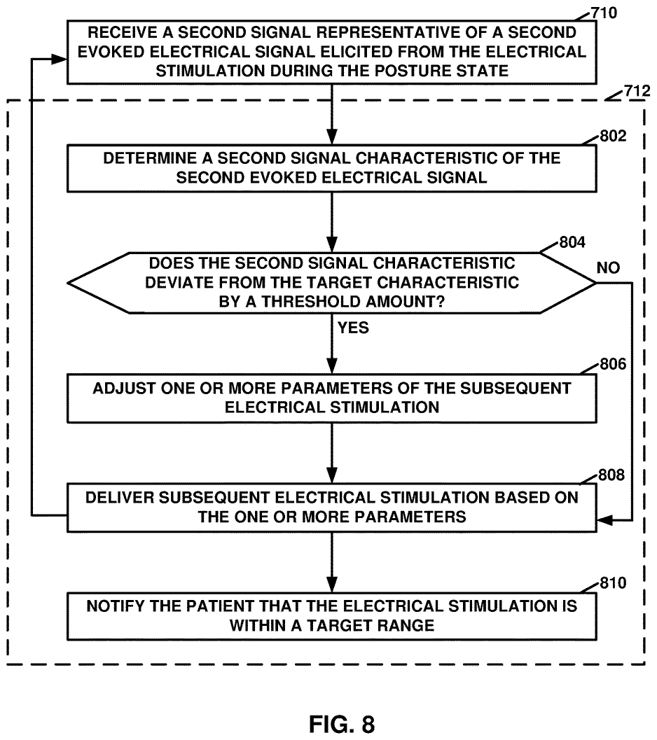

FIG. 8 illustrates an example process for controlling, based on a second signal characteristic of the second evoked electrical signal and the target characteristic(s) one or more parameters of subsequent electrical stimulation (i.e., step 712 of the example process of FIG. 7). The example process below is described primarily with reference to the example system of FIG. 1 and the example process of FIG. 7, as described above, although the example process described herein may be performed by any example device, system, or methods described in the disclosure.

IMD 106 may receive the second signal representative of the second evoked electrical signal elicited from the electrical stimulation during the posture state (710). IMD 106 may then determine a second signal characteristic of the second evoked electrical signal (802). For example, IMD 106 may determine, for the second evoked electrical signal, one or more of an amplitude of a portion of the second evoked electrical signal, a nerve conduction velocity of the second evoked electrical signal, an area under one or more peaks of the second evoked electrical signal, frequency of the second evoked electrical signal, and/or maximum and/or minimum peak timing of the second evoked electrical signal. The second signal characteristic may be a same type as one of the target characteristics for the posture state. For example, both the second signal characteristic and the target characteristic may be a frequency of the respective evoked electrical signals (e.g., the second evoked electrical signal, the baseline evoked electrical signal).

IMD 106 may determine whether second signal characteristic deviates from the target characteristic by a threshold amount (804). IMD 106 may determine whether a difference between the value of the second signal characteristic deviates from the target value(s) and/or values within a target range of the target characteristic by the threshold amount. The threshold amount may be predetermined by a clinician and transmitted to IMD 106 by external programmer 120. In some examples, based on changes over time in the signal characteristics of sensed evoked electrical signals, IMD 106 may adjust the threshold amount, e.g., to account for changes in patient physiology, changes in patient disease state, or the like.

Based on a determination that the second signal characteristic does not deviate from the target characteristic by the threshold amount (“NO” branch of 804), IMD 106 may deliver subsequent electrical stimulation based on one or more parameters (808). The values for the one or more parameters of the subsequent electrical stimulation may be similar to or the same as the values for the one or more parameters for a prior delivered electrical stimulation (e.g., electrical stimulation that elicited the second evoked electrical signal). IMD 106 may notify patient 102 that the electrical stimulation is within a target range (810). IMD 106 may continue to deliver electrical stimulation to patient 102 at the same parameter values and/or according to a same predetermined therapy stimulation program. While delivering subsequent electrical stimulation based on the one or more parameters, IMD 106 may continue to receive signals representative of evoked electrical signals elicited from the subsequent electrical stimulation during a posture state of patient 102 (710).

Based on a determination that the second signal characteristic deviates from the target characteristic by the threshold amount (“YES” branch of 804), IMD 106 may adjust one or more parameters of the subsequent electrical stimulation (806). IMD 106 may select the one or more parameters from a plurality of parameter based on predetermined instructions stored in storage device 212 of IMD 106. In some examples, IMD 106 may select the one or more parameters based on the type of the second signal characteristic. In some examples, IMD 106 may select the one or more parameters based on a magnitude of deviation between the second signal characteristic and the target characteristic.

IMD 106 may deliver subsequent electrical stimulation to tissue of patient 102 based on the one or more parameters (808). The subsequent electrical stimulation may be defined by the adjusted values of the one or more parameters and may be delivered to the same or different tissues of patient 102, depending on the adjusted values. The subsequent electrical stimulation is configured to cause patient 102 to perceive the desired comfort level while in the current posture state. IMD 106 may notify patient 102 that the electrical stimulation is within a target range (810).

IMD 106 may iteratively perform steps 710-808 of the example process of FIG. 8 until IMD 106 receives an indication from patient 102 to terminate the delivery of electrical stimulation, to temporarily cease the delivery of electrical stimulation, or to switch to another therapy stimulation program.

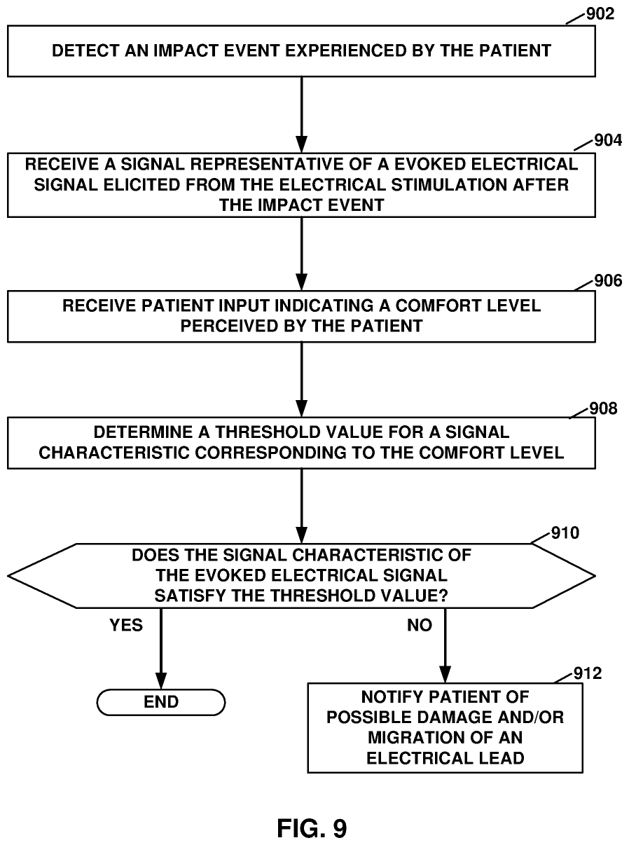

FIG. 9 is a flow diagram illustrating an example process for determining damage and/or migration of an electrical lead (e.g., leads 108) within the body of patient 102 based on a sensed evoked signal and a patient posture state, in accordance with one or more techniques of this disclosure. While FIG. 9 is described primarily with using acceleration experienced by patient 102 to determine an occurrence of an impact event (e.g., a fall, a car crash, or the like), other sensor data may be used. The other sensor data may include, but are not limited to, changes in sensor orientation and/or changes in force, pressure, and/or velocity detected by a sensor.

IMD 106 may determine an occurrence of an impact event (902). IMD 106 may detect a change in acceleration experienced by patient 102. IMD 106 may include sensor(s) 221 (e.g., an accelerometer, an IMU) configured to detect changes in acceleration experienced by IMD 106. In some examples, the sensor(s) may be coupled to patient 102 and may be in communication with IMD 106 and/or external programmer 120. IMD 106 may determine whether the magnitude of the detected acceleration satisfies a threshold condition. The threshold condition may be based on a minimum acceleration magnitude experienced by an IMD 106 for an impact event capable of damaging or moving leads 108 in patient 102. IMD 106 may determine the occurrence of an “impact event” based on a determination that the magnitude of the change in acceleration satisfies the threshold condition. IMD 106 may transmit the detected acceleration magnitude and an accompanying contextual information (e.g., a time stamp for the change in acceleration) to external programmer 120.

IMD 106 may receive a signal representative of an evoked electrical signal elicited from the electrical stimulation after the impact event (904). IMD 106 may further determine values for signal characteristics for the evoked electrical signal, e.g., in accordance with the example processes previously described in FIGS. 7 and 8.

IMD 106 may receive patient input indicating a comfort level perceived by patient 102 (906). IMD 106 may cause external programmer 120 to request patient input (e.g., via user interface 306) indicating a comfort level perceived by patient 102. The comfort level may be a current comfort level perceived by patient 102, if the request for patient input is relatively close in time to the impact event (e.g., within a number of seconds, less than one hour after the impact event). In some examples, if the request for patient input occurs after a period of time has passed since the impact event (e.g., a number of hours, a number of days). IMD 106 may request patient input indicating the comfort level perceived by patient 102 at a time immediately after the occurrence of the impact event. IMD 106 may cause external programmer 120 to provide relevant times to aid patient recall.

IMD 106 may determine a threshold condition for a signal characteristic corresponding to the comfort level (908). IMD 106 may determine, based on one or more of patient scenarios 216, a threshold value for the signal characteristic given the posture state and comfort level perceived by patient 102. For example, if patient 102 indicates a perception of the desired comfort level, the threshold value may be the target value of the target characteristic of the baseline evoked electrical signal. As another example, if patient 102 indicates a perception of a comfort level deviating from the desired comfort level by a first amount, IMD 106, may determine a value for the target characteristic based on the first amount of deviation from the target value of the target characteristic of the baseline evoked electrical signal. In some examples, IMD 106 may determine, for a given comfort level, a target range of possible values for the target characteristic.

IMD 106 may determine whether the signal characteristic of the evoked electrical signal satisfies the threshold value (910). Based on a determination that the threshold value is satisfied (“YES” branch of 910), IMD 106 may determine that the electrodes of IMD 106 are sensing expected ECAP signals given a comfort level perceived by patient 102 and no further action is taken. In such examples, IMD 106 may nonetheless repeat the example process illustrated in FIG. 9 or another lead integrity validation process at a later time, e.g., to verify the determination.

Based on a determination that the signal characteristic of the evoked electrical signal does not satisfy the threshold value (“NO” branch of 910), IMD 106 may notify patient 102 (e.g., via external programmer 120) of possible damage and/or migration of an electrical lead (912). IMD 106 may further notify a clinician of the possibility of lead damage and/or migration. In some examples IMD 106 may perform additional lead integrity validation processes, e.g., to verify the determination.

The following examples are example systems, devices, and methods as described herein.

Example 1: A system comprising: processing circuitry configured to: receive, via sensing circuitry of the system, a first signal representative of one or more first evoked electrical signals sensed from tissue of a patient and elicited from electrical stimulation delivered during a posture state of the patient; determine a first signal characteristic of the one or more first evoked electrical signals for the posture state; receive, via one or more user devices of the system, patient input indicative of a comfort level of the patient associated with the one or more first evoked electrical signals during the posture state; determine, based on the first signal characteristic and the patient input, a target characteristic for the posture state; receive, via the sensing circuitry, a second signal representative of one or more second evoked electrical signals sensed from the tissue of the patient and elicited from the electrical stimulation delivered during the posture state; and control, based on a second signal characteristic of the one or more second evoked electrical signals and the target characteristic, one or more parameters that at least partially define subsequent delivery of the electrical stimulation to the patient during the posture state.

Example 2: the system of example 1, wherein at least one of the one or more first evoked electrical signals and the one or more second evoked electrical signals comprises evoked compound action potential (ECAP) signals.

Example 3: the system of any of examples 1 and 2, wherein the processing circuitry is further configured to determine the posture state of the patient based at least in part on a signal received from an accelerometer associated with the patient.

Example 4: the system of any of examples 1-3, wherein to control the one or more parameters, the processing circuitry is further configured to: determine whether the second signal characteristic deviates from the target characteristic by a threshold amount; and based on a determination that the second signal characteristic deviates from the target characteristic by the threshold amount, adjust the one or more parameters.

Example 5: the system of example 4, wherein the processing circuitry is further configured to: sense, via the sensing circuitry, a third signal representative of one or more third evoked electrical signals sensed in the tissue and elicited from the subsequent delivery of the electrical stimulation; determine a third signal characteristic of the third evoked electrical signal; determine whether the third signal characteristic deviates from the target characteristic by the threshold amount; and based on a determination that the third signal characteristic deviates from the target characteristic by less than the threshold amount, notify the one or more user devices that the electrical stimulation is within a target range.

Example 6: the system of any of examples 4 and 5, wherein the processing circuitry is further configured to adjust the target characteristic for the posture state based at least in part on the second signal characteristic.

Example 7: the system of any of examples 1-6, wherein the patient input comprises a pain level experienced by the patient for the electrical stimulation, and wherein processing circuitry is further configured to: determine a correlation between the second signal characteristic and the patient input; and adjust the one or more parameters based at least in part on the correlation.

Example 8: the system of any of examples 1-7, wherein to control the one or more parameters, the processing circuitry is configured to: determine, based on the first signal characteristic and the patient input, a minimum stimulation amplitude to reduce pain experienced by the patient; determine one or more parameter value of the electrical stimulation based at least in part on the determined minimum amplitude; and adjust the one or more parameters of the electrical stimulation to the one or more parameter values.

Example 9: the system of any of examples 7 and 8, wherein the processing circuitry is configured to: determine a plurality of patient scenarios based on a plurality of combinations of one or more of a pain level value, a signal characteristic value, or a patient posture state from a range of possible pain level values, possible signal characteristic values, and possible patient posture states, respectively, wherein each patient scenario comprises one or more predetermined parameter value that at least partially define the electrical stimulation; select, based on the first signal characteristic and the patient input, a patient scenario of the plurality of patient scenarios, wherein each patient scenario of the plurality of patient scenarios comprises one or more predetermined parameters that at least partially define the electrical stimulation delivered to the patient; and adjust the one or more parameters to the one or more predetermined parameter values.

Example 10: the system of example 9, wherein the processing circuitry is further configured to adjust, based at least in part on the one or more second evoked electrical signals, the one or more predetermined parameter value or the minimum stimulation amplitude of one or more patient scenarios of the plurality of patient scenarios.

Example 11: the system of any of examples 9 and 10, wherein the processing circuitry is configured to: determine a minimum stimulation amplitude for each of the plurality of patient scenarios; select, based on the first signal characteristic and the patient input, the patient scenario of the plurality of patient scenarios; and adjust, based on the selected patient scenario, the one or more parameters based on the minimum stimulation amplitude of the selected patient scenario.

Example 12: the system of any of examples 1-11, wherein the patient input comprises a first patient input, and wherein the processing circuitry is further configured to: receive, via the one or more user devices, a second patient input indicating the patient is under effects of a medication; determine, based on the one or more second evoked electrical signals, an effect of the medication on the second signal characteristic; and adjust the one or more parameters based at least in part on the effect of the medication on the second signal characteristic.

Example 13: the system of example 12, wherein the second signal characteristic comprises a nerve conduction velocity of the patient, and wherein the processing circuitry is configured to determine the effect of the medication on the nerve conduction velocity of the patient.

Example 14: the system of any of examples 1-13, wherein the processing circuitry is further configured to: determine, based on the patient input, a comfort level perceived by the patient; determine whether at least one of the one or more second evoked electrical signals satisfies a threshold condition corresponding to the comfort level; and based on a determination that the at least one second evoked electrical signal satisfies the threshold condition, transmit a notification to the one or more user devices indicating migration or damage to an electrical lead coupled to a medical device, wherein the medical device is configured to deliver the electrical stimulation to the patient during the posture state.

Example 15: the system of example 14, wherein the threshold condition comprises a threshold evoked electrical signal amplitude for the patient for the determined comfort level and the posture state.

Example 16: the system of any of examples 1-15, wherein the processing circuitry is further configured to: determine, based on the patient input, that the patient is experiencing pain; and determine, based on the second signal characteristics, if changes in the one or more second evoked electrical signals from the one or more first evoked electrical signals correspond to the pain experienced by the patient.

Example 17: the system of example 16, wherein the processing circuitry is further configured to, based on a determination that the changes in the one or more second evoked electrical signals do not correspond to the pain, maintain the one or more parameters at least partially defining the electrical stimulation.

Example 18: the system of example 16, wherein the processing circuitry is further configured to, based on a determination that the changes in the one or more second evoked electrical signals do not correspond to the pain, adjust the one or more parameters at least partially defining the electrical stimulation.

Example 19: the system of example 18, wherein the processing circuitry is configured to, based on the determination that the changes in the one or more second evoked electrical signals do not correspond to the pain, adjust the one or more parameters to a predetermined level, wherein the predetermined level is configured to reduce acute pain experienced by the patient.

Example 20: the system of any of examples 1-19, wherein the first signal characteristic and the second signal characteristic each comprises a maximum amplitude of the one or more first evoked electrical signals and the one or more second evoked electrical signals, respectively.

Example 21: the system of any of examples 1-20, further comprising a stimulation generator configured to deliver electrical stimulation to the patient via one or more electrodes electrically connected to the stimulation generator, and wherein the processing circuitry is configured to control the delivery of the electrical stimulation by controlling the one or more parameters at least partially defining delivery of the electrical stimulation.

Example 22: a method comprising: receiving, by processing circuitry of a medical device system and via sensing circuitry of the medical device system, a first signal representative of one or more first evoked electrical signals sensed from tissue of a patient and elicited from electrical stimulation delivered during a posture state of the patient; determining, by the processing circuitry, a first signal characteristic of the one or more first evoked electrical signals for the posture state; receiving, by the processing circuitry from one or more user devices, patient input indicative of a comfort level of the patient associated with the one or more first evoked electrical signals during the posture state; determining, by the processing circuitry and based on the first signal characteristic and the patient input, a target characteristic for the posture; receiving, by the processing circuitry and via the sensing circuitry, a second signal representative of one or more second evoked electrical signals sensed from the tissue of the patient and elicited from the electrical stimulation delivered during the posture state; and controlling, by the processing circuitry and based on a second signal characteristic of the one or more second evoked electrical signals and the target characteristic, one or more parameters that at least partially define subsequent delivery of the electrical stimulation to the patient during the posture state.

Example 23: the method of example 22, wherein at least one of the one or more first evoked electrical signals and the one or more second evoked electrical signals comprises evoked compound action potential (ECAP) signals.

Example 24: the method of any of examples 22 and 23, further comprising determining, by the processing circuitry, the posture state of the patient based at least in part on a signal received from an accelerometer associated with the patient.

Example 25: the method of any of examples 22-24, wherein controlling the one or more parameters comprises: determining, by the processing circuitry, whether the second signal characteristic deviates from the target characteristic by a threshold amount; and adjusting, by the processing circuitry and based on a determination that the second signal characteristic deviates from the target characteristic by the threshold amount, adjust the one or more parameters.

Example 26: the method of example 25, further comprising: sensing, by the processing circuitry and via the sensing circuitry, a third signal representative of one or more third evoked electrical signals sensed in the tissue and elicited from the subsequent delivery of the electrical stimulation; determining, by the processing circuitry, a third signal characteristic of the third evoked electrical signal; determining, by the processing circuitry, whether the third signal characteristic deviates from the target characteristic by the threshold amount; and based on a determination that the one or more third signal characteristic deviates from the target characteristic by less than the threshold amount, notifying, by the processing circuitry and via the one or more user devices, the patient that the electrical stimulation is within a target range.

Example 27: the method of any of examples 25 and 26, further comprising adjusting, by the processing circuitry, the target characteristic for the posture state based at least in part on second signal characteristic.

Example 28: the method of any of examples 22-27, wherein the patient input comprises a pain level experienced by the patient for the electrical stimulation, and wherein the method further comprises: determining, by the processing circuitry, a correlation between the second signal characteristic and the patient input; and adjusting, by the processing circuitry, the one or more parameters based at least in part on the correlation.

Example 29: the method of any of examples 22-28, wherein controlling the one or more parameters comprises: determining, by the processing circuitry and based on the first signal characteristic and the patient input, a minimum stimulation amplitude to reduce pain experienced by the patient; determining, by the processing circuitry, one or more parameter value of the electrical stimulation based at least in part on the determined minimum amplitude; and adjusting, by the processing circuitry, the one or more parameters of the electrical stimulation to the one or more parameter values.

Example 30: the method of any of examples 28 and 29, further comprising: determining, by the processing circuitry, a plurality of patient scenarios based on a plurality of combinations of one or more of a pain level value, a signal characteristic value, or a patient posture state from a range of possible pain level values, possible signal characteristic values, and possible patient posture states, respectively, wherein each patient scenario comprises one or more predetermined parameter value that at least partially define the electrical stimulation; determining, by the processing circuitry, a minimum stimulation amplitude for each of the plurality of patient scenarios; selecting, by the processing circuitry and based on the first signal characteristic and the patient input, a patient scenario of the plurality of patient scenarios; and adjusting, by the processing circuitry, the one or more parameters to the one or more predetermined parameter values.

Example 31: the method of example 30, further comprising adjusting, by the processing circuitry, the one or more predetermined parameter value of one or more patient scenarios of the plurality of patient scenarios based at least in part on the second signal characteristic.

Example 32: the method of any of examples 30 and 31, further comprising: determining, by the processing circuitry, a minimum stimulation amplitude for each of the plurality of patient scenarios; selecting, by the processing circuitry and based on the first signal characteristic and the patient input, the patient scenario of the plurality of patient scenarios; and adjusting, by the processing circuitry, the one or more parameters based on the minimum stimulation amplitude of the corresponding patient scenario.

Example 33: the method of any of examples 22-32, wherein the patient input comprises a first patient input, and wherein the method further comprises: receiving, by the processing circuitry and via the one or more user devices, a second patient input indicating that the patient is under effects of a medication; determining, by the processing circuitry and based on the one or more second evoked electrical signals, an effect of the medication on the second signal characteristic; and adjusting, by the processing circuitry, the one or more based at least in part on the effect of the medication on the second signal characteristic.

Example 34: the method of examples 33, wherein the second signal characteristic comprises a nerve conduction velocity of the patient, and wherein the method further comprises: determining, by the processing circuitry, the effect of the medication on the nerve conduction velocity of the patient.

Example 35: the method of any of examples 22-34, further comprising: determining, by the processing circuitry and based on the patient input, a comfort level perceived by the patient; determining, by the processing circuitry, whether at least one of the one or more second evoked electrical signal satisfies a threshold condition corresponding to the comfort level; and based on a determination that the at least one second evoked electrical signals satisfies the threshold condition, transmitting, by the processing circuitry via the one or more user devices, a notification to patient indicating one or more of a possible migration of an electrical lead or possible damage to at least one of one or more electrodes disposed on the electrical lead.

Example 36: the method of example 35, wherein the threshold condition comprises a threshold sensed evoked electrical signal amplitude for the patient for the determined comfort level and the posture state.

Example 37: the method of any of examples 22-36, further comprising: determining, by the processing circuitry and based on the patient input, that the patient is experiencing pain; and determining, by the processing circuitry and based on the second signal characteristics, if changes in the one or more second evoked electrical signals from the one or more first evoked electrical signals correspond to the pain experienced by the patient.

Example 38: the method of example 37, further comprising maintaining, by the processing circuitry and based on a determination that the changes in the one or more second evoked electrical signals do not correspond to the pain, the one or more parameters.

Example 39: the method of example 37, further comprising adjusting, by the processing circuitry and based on a determination that the changes in the one or more second evoked electrical signals do not correspond to the pain, the one or more parameters.

Example 40: the method of example 39, further comprising adjusting, by the processing circuitry and based on the determination that the changes in the one or more second evoked electrical signals do not correspond to the pain, the one or more parameters of the electrical stimulation to one or more predetermined values, wherein the one or more predetermined value is configured to reduce an acute pain experienced by the patient.

Example 41: the method of any of examples 22-40, wherein the first signal characteristic and the second signal characteristic each comprises a maximum amplitude of the one or more first evoked electrical signals and the one or more second evoked electrical signals, respectively.

Example 42: a computer-readable medium comprising instructions that, when executed, cause processing circuitry of an implantable medical device system to perform the method of any of examples 22-41.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some respects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Further disclosed herein is the subject matter of the following examples:

Example 1: A system comprising: processing circuitry configured to: receive, via sensing circuitry of the system, a first signal representative of one or more first evoked electrical signals sensed from tissue of a patient and elicited from electrical stimulation delivered during a posture state of the patient; determine a first signal characteristic of the one or more first evoked electrical signals for the posture state; receive, via one or more user devices of the system, patient input indicative of a comfort level of the patient associated with the one or more first evoked electrical signals during the posture state; determine, based on the first signal characteristic and the patient input, a target characteristic for the posture state; receive, via the sensing circuitry, a second signal representative of one or more second evoked electrical signals sensed from the tissue of the patient and elicited from the electrical stimulation delivered during the posture state; and control, based on a second signal characteristic of the one or more second evoked electrical signals and the target characteristic, one or more parameters that at least partially define subsequent delivery of the electrical stimulation to the patient during the posture state.

Example 2: The system of claim 1, wherein at least one of the one or more first evoked electrical signals and the one or more second evoked electrical signals comprises evoked compound action potential (ECAP) signals.

Example 3: The system of any of claims 1 and 2, wherein the processing circuitry is further configured to determine the posture state of the patient based at least in part on a signal received from an accelerometer associated with the patient.

Example 4: The system of any of claims 1-3, wherein to control the one or more parameters, the processing circuitry is further configured to: determine whether the second signal characteristic deviates from the target characteristic by a threshold amount; and based on a determination that the second signal characteristic deviates from the target characteristic by the threshold amount, adjust the one or more parameters.

Example 5: The system of claim 4, wherein the processing circuitry is further configured to: sense, via the sensing circuitry, a third signal representative of one or more third evoked electrical signals sensed in the tissue and elicited from the subsequent delivery of the electrical stimulation; determine a third signal characteristic of the third evoked electrical signal; determine whether the third signal characteristic deviates from the target characteristic by the threshold amount; and based on a determination that the third signal characteristic deviates from the target characteristic by less than the threshold amount, notify the one or more user devices that the electrical stimulation is within a target range.

Example 6: The system of any of claims 4 and 5, wherein the processing circuitry is further configured to adjust the target characteristic for the posture state based at least in part on the second signal characteristic.

Example 7: The system of any of claims 1-6, wherein the patient input comprises a pain level experienced by the patient for the electrical stimulation, and wherein processing circuitry is further configured to: determine a correlation between the second signal characteristic and the patient input; and adjust the one or more parameters based at least in part on the correlation.

Example 8: The system of any of claims 1-7, wherein to control the one or more parameters, the processing circuitry is configured to: determine, based on the first signal characteristic and the patient input, a minimum stimulation amplitude to reduce pain experienced by the patient; determine one or more parameter value of the electrical stimulation based at least in part on the determined minimum amplitude; and adjust the one or more parameters of the electrical stimulation to the one or more parameter values.

Example 9: The system of any of claims 7 and 8, wherein the processing circuitry is configured to: determine a plurality of patient scenarios based on a plurality of combinations of one or more of a pain level value, a signal characteristic value, or a patient posture state from a range of possible pain level values, possible signal characteristic values, and possible patient posture states, respectively, wherein each patient scenario comprises one or more predetermined parameter value that at least partially define the electrical stimulation; select, based on the first signal characteristic and the patient input, a patient scenario of the plurality of patient scenarios, wherein each patient scenario of the plurality of patient scenarios comprises one or more predetermined parameters that at least partially define the electrical stimulation delivered to the patient; and adjust the one or more parameters to the one or more predetermined parameter values.

Example 10: The system of claim 9, wherein the processing circuitry is further configured to adjust, based at least in part on the one or more second evoked electrical signals, the one or more predetermined parameter value or the minimum stimulation amplitude of one or more patient scenarios of the plurality of patient scenarios.

Example 11: The system of any of claims 9 and 10, wherein the processing circuitry is configured to: determine a minimum stimulation amplitude for each of the plurality of patient scenarios; select, based on the first signal characteristic and the patient input, the patient scenario of the plurality of patient scenarios; and adjust, based on the selected patient scenario, the one or more parameters based on the minimum stimulation amplitude of the selected patient scenario.

Example 12: The system of any of claims 1-11, wherein the patient input comprises a first patient input, and wherein the processing circuitry is further configured to: receive, via the one or more user devices, a second patient input indicating the patient is under effects of a medication; determine, based on the one or more second evoked electrical signals, an effect of the medication on the second signal characteristic; and adjust the one or more parameters based at least in part on the effect of the medication on the second signal characteristic.

Example 13: The system of claim 12, wherein the second signal characteristic comprises a nerve conduction velocity of the patient, and wherein the processing circuitry is configured to determine the effect of the medication on the nerve conduction velocity of the patient.

Example 14: The system of any of claims 1-13, wherein the processing circuitry is further configured to: determine, based on the patient input, a comfort level perceived by the patient; determine whether at least one of the one or more second evoked electrical signals satisfies a threshold condition corresponding to the comfort level; and based on a determination that the at least one second evoked electrical signal satisfies the threshold condition, transmit a notification to the one or more user devices indicating migration or damage to an electrical lead coupled to a medical device, wherein the medical device is configured to deliver the electrical stimulation to the patient during the posture state.

Example 15: The system of claim 14, wherein the threshold condition comprises a threshold evoked electrical signal amplitude for the patient for the determined comfort level and the posture state.

Example 16: The system of any of claims 1-15, wherein the processing circuitry is further configured to: determine, based on the patient input, that the patient is experiencing pain; and determine, based on the second signal characteristics, if changes in the one or more second evoked electrical signals from the one or more first evoked electrical signals correspond to the pain experienced by the patient.

Example 17: The system of claim 16, wherein the processing circuitry is further configured to, based on a determination that the changes in the one or more second evoked electrical signals do not correspond to the pain, maintain the one or more parameters at least partially defining the electrical stimulation.

Example 18: The system of claim 16, wherein the processing circuitry is further configured to, based on a determination that the changes in the one or more second evoked electrical signals do not correspond to the pain, adjust the one or more parameters at least partially defining the electrical stimulation.

Example 19: The system of claim 18, wherein the processing circuitry is configured to, based on the determination that the changes in the one or more second evoked electrical signals do not correspond to the pain, adjust the one or more parameters to a predetermined level, wherein the predetermined level is configured to reduce acute pain experienced by the patient.

Example 20: The system of any of claims 1-19, wherein the first signal characteristic and the second signal characteristic each comprises a maximum amplitude of the one or more first evoked electrical signals and the one or more second evoked electrical signals, respectively.

Example 21: The system of any of claims 1-20, further comprising a stimulation generator configured to deliver electrical stimulation to the patient via one or more electrodes electrically connected to the stimulation generator, and wherein the processing circuitry is configured to control the delivery of the electrical stimulation by controlling the one or more parameters at least partially defining delivery of the electrical stimulation.

Example 22: A method comprising: receiving, by processing circuitry of a medical device system and via sensing circuitry of the medical device system, a first signal representative of one or more first evoked electrical signals sensed from tissue of a patient and elicited from electrical stimulation delivered during a posture state of the patient; determining, by the processing circuitry, a first signal characteristic of the one or more first evoked electrical signals for the posture state; receiving, by the processing circuitry from one or more user devices, patient input indicative of a comfort level of the patient associated with the one or more first evoked electrical signals during the posture state; determining, by the processing circuitry and based on the first signal characteristic and the patient input, a target characteristic for the posture; receiving, by the processing circuitry and via the sensing circuitry, a second signal representative of one or more second evoked electrical signals sensed from the tissue of the patient and elicited from the electrical stimulation delivered during the posture state; and controlling, by the processing circuitry and based on a second signal characteristic of the one or more second evoked electrical signals and the target characteristic, one or more parameters that at least partially define subsequent delivery of the electrical stimulation to the patient during the posture state.

Example 23: The method of claim 22, wherein at least one of the one or more first evoked electrical signals and the one or more second evoked electrical signals comprises evoked compound action potential (ECAP) signals.

Example 24: The method of any of claims 22 and 23, further comprising determining, by the processing circuitry, the posture state of the patient based at least in part on a signal received from an accelerometer associated with the patient.

Example 25: The method of any of claims 22-24, wherein controlling the one or more parameters comprises: determining, by the processing circuitry, whether the second signal characteristic deviates from the target characteristic by a threshold amount; and adjusting, by the processing circuitry and based on a determination that the second signal characteristic deviates from the target characteristic by the threshold amount, adjust the one or more parameters.

Example 26: The method of claim 25, further comprising: sensing, by the processing circuitry and via the sensing circuitry, a third signal representative of one or more third evoked electrical signals sensed in the tissue and elicited from the subsequent delivery of the electrical stimulation; determining, by the processing circuitry, a third signal characteristic of the third evoked electrical signal; determining, by the processing circuitry, whether the third signal characteristic deviates from the target characteristic by the threshold amount; and based on a determination that the one or more third signal characteristic deviates from the target characteristic by less than the threshold amount, notifying, by the processing circuitry and via the one or more user devices, the patient that the electrical stimulation is within a target range.

Example 27: The method of any of claims 25 and 26, further comprising adjusting, by the processing circuitry, the target characteristic for the posture state based at least in part on second signal characteristic.

Example 28: The method of any of claims 22-27, wherein the patient input comprises a pain level experienced by the patient for the electrical stimulation, and wherein the method further comprises: determining, by the processing circuitry, a correlation between the second signal characteristic and the patient input; and adjusting, by the processing circuitry, the one or more parameters based at least in part on the correlation.

Example 29: The method of any of claims 22-28, wherein controlling the one or more parameters comprises: determining, by the processing circuitry and based on the first signal characteristic and the patient input, a minimum stimulation amplitude to reduce pain experienced by the patient; determining, by the processing circuitry, one or more parameter value of the electrical stimulation based at least in part on the determined minimum amplitude; and adjusting, by the processing circuitry, the one or more parameters of the electrical stimulation to the one or more parameter values.

Example 30: The method of any of claims 28 and 29, further comprising: determining, by the processing circuitry, a plurality of patient scenarios based on a plurality of combinations of one or more of a pain level value, a signal characteristic value, or a patient posture state from a range of possible pain level values, possible signal characteristic values, and possible patient posture states, respectively, wherein each patient scenario comprises one or more predetermined parameter value that at least partially define the electrical stimulation; determining, by the processing circuitry, a minimum stimulation amplitude for each of the plurality of patient scenarios; selecting, by the processing circuitry and based on the first signal characteristic and the patient input, a patient scenario of the plurality of patient scenarios; and adjusting, by the processing circuitry, the one or more parameters to the one or more predetermined parameter values.

Example 31: The method of claim 30, further comprising adjusting, by the processing circuitry, the one or more predetermined parameter value of one or more patient scenarios of the plurality of patient scenarios based at least in part on the second signal characteristic.

Example 32: The method of any of claims 30 and 31, further comprising: determining, by the processing circuitry, a minimum stimulation amplitude for each of the plurality of patient scenarios; selecting, by the processing circuitry and based on the first signal characteristic and the patient input, the patient scenario of the plurality of patient scenarios; and adjusting, by the processing circuitry, the one or more parameters based on the minimum stimulation amplitude of the corresponding patient scenario.

Example 33: The method of any of claims 22-32, wherein the patient input comprises a first patient input, and wherein the method further comprises: receiving, by the processing circuitry and via the one or more user devices, a second patient input indicating that the patient is under effects of a medication; determining, by the processing circuitry and based on the one or more second evoked electrical signals, an effect of the medication on the second signal characteristic; and adjusting, by the processing circuitry, the one or more based at least in part on the effect of the medication on the second signal characteristic.

Example 34: The method of claim 33, wherein the second signal characteristic comprises a nerve conduction velocity of the patient, and wherein the method further comprises: determining, by the processing circuitry, the effect of the medication on the nerve conduction velocity of the patient.

Example 35: The method of any of claims 22-34, further comprising: determining, by the processing circuitry and based on the patient input, a comfort level perceived by the patient; determining, by the processing circuitry, whether at least one of the one or more second evoked electrical signal satisfies a threshold condition corresponding to the comfort level; and based on a determination that the at least one second evoked electrical signals satisfies the threshold condition, transmitting, by the processing circuitry via the one or more user devices, a notification to patient indicating one or more of a possible migration of an electrical lead or possible damage to at least one of one or more electrodes disposed on the electrical lead.

Example 36: The method of claim 35, wherein the threshold condition comprises a threshold sensed evoked electrical signal amplitude for the patient for the determined comfort level and the posture state.

Example 37: The method of any of claims 22-36, further comprising: determining, by the processing circuitry and based on the patient input, that the patient is experiencing pain; and determining, by the processing circuitry and based on the second signal characteristics, if changes in the one or more second evoked electrical signals from the one or more first evoked electrical signals correspond to the pain experienced by the patient.

Example 38: The method of claim 37, further comprising maintaining, by the processing circuitry and based on a determination that the changes in the one or more second evoked electrical signals do not correspond to the pain, the one or more parameters.

Example 39: The method of claim 37, further comprising adjusting, by the processing circuitry and based on a determination that the changes in the one or more second evoked electrical signals do not correspond to the pain, the one or more parameters.

Example 40: The method of claim 39, further comprising adjusting, by the processing circuitry and based on the determination that the changes in the one or more second evoked electrical signals do not correspond to the pain, the one or more parameters of the electrical stimulation to one or more predetermined values, wherein the one or more predetermined value is configured to reduce an acute pain experienced by the patient.

Example 41: The method of any of claims 22-40, wherein the first signal characteristic and the second signal characteristic each comprises a maximum amplitude of the one or more first evoked electrical signals and the one or more second evoked electrical signals, respectively.

Example 42: A computer-readable medium comprising instructions that, when executed, cause processing circuitry of an implantable medical device system to perform the method of any of claims 22-41.

Various examples have been described. These and other examples are within the scope of the following claims.

1-15. (canceled)

16. A system comprising:
processing circuitry configured to:
receive, via sensing circuitry of the system, a first signal representative of one or more first evoked electrical signals sensed from tissue of a patient and elicited from electrical stimulation delivered during a posture state of the patient;
determine a first signal characteristic of the one or more first evoked electrical signals for the posture state;
receive, via one or more user devices of the system, patient input indicative of a comfort level of the patient associated with the one or more first evoked electrical signals during the posture state;
determine, based on the first signal characteristic and the patient input, a target characteristic for the posture state;
receive, via the sensing circuitry, a second signal representative of one or more second evoked electrical signals sensed from the tissue of the patient and elicited from the electrical stimulation delivered during the posture state; and
control, based on a second signal characteristic of the one or more second evoked electrical signals and the target characteristic, one or more parameters that at least partially define subsequent delivery of the electrical stimulation to the patient during the posture state.

17. The system of claim 16, wherein at least one of the one or more first evoked electrical signals and the one or more second evoked electrical signals comprises evoked compound action potential (ECAP) signals.

18. The system of claim 16, wherein the processing circuitry is further configured to determine the posture state of the patient based at least in part on a signal received from an accelerometer associated with the patient.

19. The system of claim 16, wherein to control the one or more parameters, the processing circuitry is further configured to:
determine whether the second signal characteristic deviates from the target characteristic by a threshold amount; and
based on a determination that the second signal characteristic deviates from the target characteristic by the threshold amount, adjust the one or more parameters.

20. The system of claim 19, wherein the processing circuitry is further configured to:
sense, via the sensing circuitry, a third signal representative of one or more third evoked electrical signals sensed in the tissue and elicited from the subsequent delivery of the electrical stimulation;
determine a third signal characteristic of the third evoked electrical signal;
determine whether the third signal characteristic deviates from the target characteristic by the threshold amount; and
based on a determination that the third signal characteristic deviates from the target characteristic by less than the threshold amount, notify the one or more user devices that the electrical stimulation is within a target range.

21. The system of claim 16, wherein the patient input comprises a pain level experienced by the patient for the electrical stimulation, and wherein processing circuitry is further configured to:
determine a correlation between the second signal characteristic and the patient input; and
adjust the one or more parameters based at least in part on the correlation.

22. The system of claim 16, wherein to control one or more parameters, the processing circuitry is configured to:
determine, based on the first signal characteristic and the patient input, a minimum stimulation amplitude to reduce pain experienced by the patient;
determine one or more parameter value of the electrical stimulation based at least in part on the determined minimum amplitude; and
adjust the one or more parameters of the electrical stimulation to the one or more parameter values.

23. The system of claim 22, wherein the processing circuitry is configured to:
determine a plurality of patient scenarios based on a plurality of combinations of one or more of a pain level value, a signal characteristic value, or a patient posture state from a range of possible pain level values, possible signal characteristic values, and possible patient posture states, respectively, wherein each patient scenario comprises one or more predetermined parameter value that at least partially define the electrical stimulation;
select, based on the first signal characteristic and the patient input, a patient scenario of the plurality of patient scenarios, wherein each patient scenario of the plurality of patient scenarios comprises one or more predetermined parameters that at least partially define the electrical stimulation delivered to the patient; and
adjust the one or more parameters to the one or more predetermined parameter values.

24. The system of claim 23, wherein the processing circuitry is configured to:
determine a minimum stimulation amplitude for each of the plurality of patient scenarios;
select, based on the first signal characteristic and the patient input, the patient scenario of the plurality of patient scenarios; and
adjust, based on the selected patient scenario, the one or more parameters based on the minimum stimulation amplitude of the selected patient scenario.

25. The system of claim 16, wherein the patient input comprises a first patient input, and wherein the processing circuitry is further configured to:
receive, via the one or more user devices, a second patient input indicating the patient is under effects of a medication;
determine, based on the one or more second evoked electrical signals, an effect of the medication on the second signal characteristic; and
adjust the one or more parameters based at least in part on the effect of the medication on the second signal characteristic.

26. A method comprising:
receiving, by processing circuitry of a medical device system and via sensing circuitry of the medical device system, a first signal representative of one or more first evoked electrical signals sensed from tissue of a patient and elicited from electrical stimulation delivered during a posture state of the patient;
determining, by the processing circuitry, a first signal characteristic of the one or more first evoked electrical signals for the posture state;
receiving, by the processing circuitry from one or more user devices, patient input indicative of a comfort level of the patient associated with the one or more first evoked electrical signals during the posture state;
determining, by the processing circuitry and based on the first signal characteristic and the patient input, a target characteristic for the posture;
receiving, by the processing circuitry and via the sensing circuitry, a second signal representative of one or more second evoked electrical signals sensed from the tissue of the patient and elicited from the electrical stimulation delivered during the posture state; and
controlling, by the processing circuitry and based on a second signal characteristic of the one or more second evoked electrical signals and the target characteristic, one or more parameters that at least partially define subsequent delivery of the electrical stimulation to the patient during the posture state.

27. The method of claim 26, wherein at least one of the one or more first evoked electrical signals and the one or more second evoked electrical signals comprises evoked compound action potential (ECAP) signals.

28. The method of claim 26, wherein controlling the one or more parameters comprises:
determining, by the processing circuitry, whether the second signal characteristic deviates from the target characteristic by a threshold amount; and
adjusting, by the processing circuitry and based on a determination that the second signal characteristic deviates from the target characteristic by the threshold amount, adjust the one or more parameters.

29. The method of claim 28, further comprising:
sensing, by the processing circuitry and via the sensing circuitry, a third signal representative of one or more third evoked electrical signals sensed in the tissue and elicited from the subsequent delivery of the electrical stimulation;
determining, by the processing circuitry, a third signal characteristic of the third evoked electrical signal;
determining, by the processing circuitry, whether the third signal characteristic deviates from the target characteristic by the threshold amount; and
based on a determination that the one or more third signal characteristic deviates from the target characteristic by less than the threshold amount, notifying, by the processing circuitry and via the one or more user devices, the patient that the electrical stimulation is within a target range.

30. The method of claim 26, wherein the patient input comprises a pain level experienced by the patient for the electrical stimulation, and wherein the method further comprises:
determining, by the processing circuitry, a correlation between the second signal characteristic and the patient input; and
adjusting, by the processing circuitry, the one or more parameters based at least in part on the correlation.

31. The method of claim 26, wherein controlling the one or more parameters comprises:
determining, by the processing circuitry and based on the first signal characteristic and the patient input, a minimum stimulation amplitude to reduce pain experienced by the patient;
determining, by the processing circuitry, one or more parameter value of the electrical stimulation based at least in part on the determined minimum amplitude; and
adjusting, by the processing circuitry, the one or more parameters of the electrical stimulation to the one or more parameter values.

32. A computer-readable medium comprising instructions that, when executed, cause processing circuitry of an implantable medical device system to:
receive, via sensing circuitry of the medical device system, a first signal representative of one or more first evoked electrical signals sensed from tissue of a patient and elicited from electrical stimulation delivered during a posture state of the patient;
determine a first signal characteristic of the one or more first evoked electrical signals for the posture state;
receive, from one or more user devices, patient input indicative of a comfort level of the patient associated with the one or more first evoked electrical signals during the posture state;
determine, based on the first signal characteristic and the patient input, a target characteristic for the posture;
receive, via the sensing circuitry, a second signal representative of one or more second evoked electrical signals sensed from the tissue of the patient and elicited from the electrical stimulation delivered during the posture state; and
control, based on a second signal characteristic of the one or more second evoked electrical signals and the target characteristic, one or more parameters that at least partially define subsequent delivery of the electrical stimulation to the patient during the posture state.

33. The computer-readable medium of claim 32, wherein at least one of the one or more first evoked electrical signals and the one or more second evoked electrical signals comprises evoked compound action potential (ECAP) signals.

34. The computer-readable medium of claim 32, further comprising instructions that, when executed, causes the processing circuitry to control the one or more parameters by:
determining whether the second signal characteristic deviates from the target characteristic by a threshold amount; and
adjusting, based on a determination that the second signal characteristic deviates from the target characteristic by the threshold amount, adjust the one or more parameters.

35. The computer-readable medium of claim 32, further comprising instructions that, when executed, causes the processing circuitry to:
sense, via the sensing circuitry, a third signal representative of one or more third evoked electrical signals sensed in the tissue and elicited from the subsequent delivery of the electrical stimulation;
determine a third signal characteristic of the third evoked electrical signal;
determine whether the third signal characteristic deviates from the target characteristic by the threshold amount; and
based on a determination that the one or more third signal characteristic deviates from the target characteristic by less than the threshold amount, notify, via the one or more user devices, the patient that the electrical stimulation is within a target range.
